(19) United States
(12) Patent Application Publication
(10) Pub. No.: US 2004/0072770 A1
(43) Pub. Date: Apr. 15, 2004

(54) Methods for specifically inhibiting histone deacetylase-7 and 8

(76) Inventors: Jeffrey M. Besterman, Bai D'urfe (CA); Zuomei Li, Kirkland (CA); Daniel Delorme, St-Lazare (CA); Claire Bonfils, Montreal (CA)

Correspondence Address: WAYNE A. KEOWN, SUITE 1200, 500 WEST CUMMINGS PARK, WOBURN, MA, 01801, US

(21) Appl. No.: 10/189,818

(22) Filed: Jul. 3, 2002

(51) Int. Cl. A61K 48/00; C07H 21/04

(52) U.S. Cl. 514/44; 536/23.5

(57) ABSTRACT

This invention relates to the inhibition of histone deacetylase (HDAC) expression and enzymatic activity. The invention provides methods and reagents for inhibiting HDAC-7 and HDAC-8 by inhibiting expression at the nucleic acid level or inhibiting enzymatic activity at the protein level.

### BACKGROUND OF THE INVENTION

1. Field of the Invention

The present invention relates to the fields of molecular biology and medicine. More specifically, the invention relates to the fields of gene expression and oncology.

2. Summary of the Related Art

Chromatin is the complex of proteins and DNA in the nucleus of eukaryotes. Chromatin proteins provide structural and functional organization to nuclear DNA. The nucleosome is the fundamental unit of structural organization of chromatin. The nucleosome principally consists of (1) the core histones, termed H2A, H2B, H3, and H4, which associate to form a protein core particle, and (2) the approximately 146 base pairs of DNA wrapped around the histone core particle. The physical interaction between the core histone particle and DNA principally occurs through the negatively charged phosphate groups of the DNA and the basic amino acid moieties of the histone proteins. (Csordas, Biochem. J, 286:23-38 (1990)) teaches that histones are subject to posttranslational acetylation of their epsilon-amino groups of N-terminal lysine residues, a reaction that is catalyzed by histone acetyl transferase (HAT). The posttranslational acetylation of histones has both structural and functional, i.e., gene regulatory, consequences.

Acetylation neutralizes the positive charge of the epsilon-amino groups of N-terminal lysine residues, thereby influencing the interaction of DNA with the histone core particle of the nucleosome. Thus, histone acetylation and histone deacetylation (HDAC) are thought to impact chromatin structure and gene regulation. For example, Taunton et al., Science, 272:408-411 (1996), teaches that access of transcription factors to chromatin templates is enhanced by histone hyperacetylation. Taunton et al. further teaches that an enrichment in underacetylated histone H4 has been found in transcriptionally silent regions of the genome.

Studies utilizing known HDAC inhibitors have established a link between acetylation and gene expression. Yoshida et al, Cancer Res. 47:3688-3691 (1987) discloses that (R)-Trichostatin A (TSA) is a potent inducer of differentiation in murine erythroleukemia cells. Yoshida et al., J. Biol. Chem. 265:17174-17179 (1990) teaches that TSA is a potent inhibitor of mammalian HDAC.

Numerous studies have examined the relationship between HDAC and gene expression. Taunton et al., Science 272:408-411 (1996), discloses a human HDAC that is related to a yeast transcriptional regulator. Cress et al., J. Cell. Phys. 184:1-16 (2000), discloses that, in the context of human cancer, the role of HDAC is as a corepressor of transcription. Ng et al., TIBS 25: March (2000), discloses HDAC as a pervasive feature of transcriptional repressor systems. Magnaghi-Jaulin et al., Prog. Cell Cycle Res. 4:41-47 (2000), discloses HDAC as a transcriptional co-regulator important for cell cycle progression.

The molecular cloning of gene sequences encoding proteins with HDAC activity has established the existence of a set of discrete HDAC enzyme isoforms. Grozinger et al., Proc. Natl. Acad. Sci. USA, 96:4868-4873 (1999), teaches that HDACs may be divided into two classes, the first represented by yeast Rpd3 -like proteins, and the second represented by yeast Hd1-like proteins. Grozinger et al. also teaches that the human HDAC-1, HDAC-2, and HDAC-3 proteins are members of the first class of HDACs, and discloses new proteins, named HDAC-4, HDAC-5, and HDAC-6, which are members of the second class of HDACs. Kao et al., Gene & Development 14:55-66 (2000), discloses an additional member of this second class, called HDAC-7. More recently, Hu, E. et al. J. Bio. Chem. 275:15254-13264 (2000) discloses the newest member of the first class of histone deacetylases, HDAC-8. Zhou et al., Proc. Natl. Acad. Sci. U.S.A., 98: 10572-10577 (2001) teaches the cloning and characterization of a new histone deacetylase, HDAC-9. Kao et al., J. Biol. Chem., 277:187-93 (2002) teaches the isolation and characterization of mammalian HDAC10, a novel histone deacetylase. Gao et al, . J. Biol. Chem. (In press) teaches the cloning and functional characterization of HDAC11, a novel member of the human histone deacetylase family. Shore, Proc. Natl. Acad. Sci. U.S.A. 97: 14030-2 (2000) discloses a third class of deacetylase activity, the Sir2 protein family. It has been unclear what roles these individual HDAC enzymes play.

Known inhibitors of mammalian HDAC have been used to probe the role of HDAC in gene regulation for some time. Yoshida et al., J. Biol. Chem. 265:17174-17179 (1990) discloses that (R)-Trichostatin A (TSA) is a potent inhibitor of mammalian HDAC. Yoshida et al, Cancer Res. 47:3688-3691 (1987) discloses that TSA is a potent inducer of differentiation in murine erythroleukemia cells.

Known inhibitors of histone deacetylase are all small molecules that inhibit histone deacetylase activity at the protein level. Moreover, all of the known histone deacetylase inhibitors are non-specific for a particular histone deacetylase isoform, and more or less inhibit all members of both the histone deacetylase families equally. (Grozinger, C. M., et al., Proc. Natl. Acad. Sci. U.S.A. 96:4868-4873 (1999)). For example, see Marks et al., J. National Cancer Inst. 92:1210-1216 (2000), which reviews histone deacetylase inhibitors and their role in studying differentiation and apoptosis.

Therefore, there remains a need to develop reagents for inhibiting specific histone deacetylase isoforms. There is also a need for the development of methods for using these reagents to modulate the activity of specific histone deacetylase isoforms and to identify those isoforms involved in tumorigenesis and other proliferative diseases and disorders.

### BRIEF SUMMARY OF THE INVENTION

The invention provides methods and reagents for modulating the activity of histone deacetylase (HDAC) isoforms. For example, the invention provides methods and reagents for inhibiting HCAC isoforms, particularly HDAC-7 and HDAC-8, by inhibiting expression at the nucleic acid level or enzymatic activity at the protein level. The invention provides for the specific inhibition of specific histone deacetylase isoforms involved in tumorigenesis and thus provides a treatment for cancer. The invention further provides for the specific inhibition of particular HDAC isoforms involved in cell proliferation, and thus provides a treatment for cell proliferative diseases and disorders.

The inventors have made the surprising discovery that the specific inhibition of HDAC-7 and 8 dramatically induce apoptosis and/or growth arrest in cancerous cells. Accordingly, in a first aspect, the invention provides agents that inhibit the activity of the HDAC-7 and HDAC-8 isoforms.

In certain preferred embodiments of the first aspect of the invention, the agent that inhibits the HDAC-7 and HDAC-8 isoforms is an oligonucleotide that inhibits expression of a nucleic acid molecule encoding the HDAC-7 and HDAC-8 isoforms. The nucleic acid molecule encoding the HDAC-7 and HDAC-8 isoforms may be genomic DNA (e.g., a gene), cDNA, or RNA. In some embodiments, the oligonucleotide inhibits transcription of mRNA encoding the HDAC-7 or HDAC-8 isoforms. In other embodiments, the oligonucleotide inhibits translation of the HDAC-7 or HDAC-8 isoforms. In certain embodiments the oligonucleotide causes the degradation of the nucleic acid molecule.

In a preferred embodiment thereof, the agent of the first aspect of the invention is an antisense oligonucleotide complementary to a region of RNA that encodes a portion of HDAC-7 or HDAC-8 or to a region of double-stranded DNA that encodes a portion of HDAC-7 or HDAC-8 isoforms. In one embodiment thereof, the antisense oligonucleotide is a chimeric oligonucleotide. In another embodiment thereof, the antisense oligonucleotide is a hybrid oligonucleotide. In another embodiment thereof, the antisense oligonucleotide has a nucleotide sequence of from about 13 to about 35 nucleotides selected from the nucleotide sequence of SEQ ID NO: 1. In still yet another embodiment thereof, the antisense oligonucleotide has a nucleotide sequence of from about 15 to about 26 nucleotides selected from the nucleotide sequence of SEQ ID NO: 1. In another embodiment thereof, the antisense oligonucleotide has a nucleotide sequence of from about 20 to about 26 nucleotides selected from the nucleotide sequence of SEQ ID NO: 1. In another embodiment thereof, the antisense oligonucleotide has a nucleotide sequence of from about 13 to about 35 nucleotides and which comprises the nucleotide sequence of SEQ ID NO: 2. In still yet another embodiment thereof, the antisense oligonucleotide has a nucleotide sequence of from about 15 to about 26 nucleotides and which comprises the nucleotide sequence of SEQ ID NO: 2. In another embodiment thereof, the antisense oligonucleotide has a nucleotide sequence of from about 20 to about 26 nucleotides and which comprises the nucleotide sequence of SEQ ID NO: 2. In another embodiment thereof, the antisense oligonucleotide is SEQ ID NO: 3. In another embodiment thereof, the antisense oligonucleotide has one or more phosphorothioate internucleoside linkages. In another embodiment thereof, the antisense oligonucleotide further comprises a length of 20-26 nucleotides. In still another embodiment thereof, the antisense oligonucleotide is modified such that the terminal four nucleotides at the 5′ end of the oligonucleotide and the terminal four nucleotides at the 3′ end of the oligonucleotide each have 2′-O-methyl groups attached to their sugar residues.

In certain preferred embodiments of the first aspect, the agent that inhibits the HDAC-7 and/or HDAC-8 isoform in a cell is a small molecule inhibitor that inhibits expression of a nucleic acid molecule encoding HDAC-7 or HDAC-8 isoform or activity of the HDAC-7 and/or HDAC-8 protein.

In a second aspect, the invention provides a method for inhibiting HDAC-7 and/or HDAC-8 activity in a cell, comprising contacting the cell with a specific inhibitor of HDAC-7 and/or HDAC-8, whereby HDAC-7 and/or HDAC-8 activity is inhibited. In an embodiment thereof, the invention provides method for inhibiting the HDAC-7 or HDAC-8 isoform in a cell, comprising contacting the cell with an antisense oligonucleotide complementary to a region of RNA that encodes a portion of HDAC-7 or HDAC-8 or to a region of double-stranded DNA that encodes a portion of HDAC-7 or HDAC-8, whereby HDAC-7 or HDAC-8 activity is inhibited. In one embodiment thereof, the cell is contacted with an HDAC-7 or HDAC-8 antisense oligonucleotide that is a chimeric oligonucleotide. In another embodiment thereof, the cell is contacted with an HDAC-7 or HDAC-8 antisense oligonucleotide that is a hybrid oligonucleotide. In another embodiment thereof, the antisense oligonucleotide has a nucleotide sequence of from about 13 to about 35 nucleotides selected from the nucleotide sequence of SEQ ID NO: 1. In still yet another embodiment thereof, the antisense oligonucleotide has a nucleotide sequence of from about 15 to about 26 nucleotides selected from the nucleotide sequence of SEQ ID NO: 1. In another embodiment thereof, the antisense oligonucleotide has a nucleotide sequence of from about 20 to about 26 nucleotides selected from the nucleotide sequence of SEQ ID NO: 1. In yet another embodiment thereof, the cell is contacted with an HDAC-7 antisense oligonucleotide that has a nucleotide sequence length of from about 13 to about 35 nucleotides and which comprises the nucleotide sequence of SEQ ID NO: 3. In another embodiment thereof, the cell is contacted with an HDAC-8 antisense oligonucleotide that has a nucleotide sequence length of from about 15 to about 26 nucleotides and which comprises the nucleotide sequence of SEQ ID NO: 4. In another embodiment thereof, the cell is contacted with an HDAC-8 antisense oligonucleotide that is SEQ ID NO: 4. In another embodiment thereof, the inhibition of HDAC-7 or HDAC-8 activity leads to the inhibition of cell proliferation in the contacted cell. In another embodiment thereof, the inhibition of HDAC-7 or HDAC-8 activity in the contacted cell further leads to growth retardation of the contacted cell. In another embodiment thereof, the inhibition of HDAC-7 or HDAC-8 activity in the contacted cell further leads to growth arrest of the contacted cell. In another embodiment thereof, the inhibition of HDAC-7 or HDAC-8 activity in the contacted cell further leads to programmed cell death of the contacted cell. In another embodiment thereof, the inhibition of HDAC-7 or HDAC-8 activity in the contacted cell further leads to necrotic cell death of the contacted cell. In certain embodiments thereof, the cell is a neoplastic cell which may be in an animal, including a human, and which may be in a neoplastic growth. In certain preferred embodiments, the method further comprises contacting the cell with an HDAC-7 and/or HDAC-8 small molecule inhibitor that interacts with and reduces the enzymatic activity of the HDAC-7 and or HDAC-8 histone deacetylase isoform. In some embodiments thereof, the histone deacetylase small molecule inhibitor is operably associated with the antisense oligonucleotide.

In a third aspect, the invention provides a method for inhibiting neoplastic cell proliferation in an animal, comprising administering to an animal having at least one neoplastic cell present in its body a therapeutically effective amount of a specific inhibitor of HDAC-7 and/or HDAC-8, whereby neoplastic cell proliferation is inhibited in the animal. In an embodiment thereof, the invention provides a method for inhibiting neoplastic cell growth in an animal, comprising administering to an animal having at least one neoplastic cell present in its body a therapeutically effective amount of the antisense oligonucleotide of the first aspect of the invention with a pharmaceutically acceptable carrier for a therapeutically effective period of time. In an embodiment thereof, the animal is administered a chimeric HDAC-7 or antisense oligonucleotide. In another embodiment thereof, the animal is administered a hybrid HDAC-7 or HDAC-8 antisense oligonucleotide. In another embodiment thereof, the antisense oligonucleotide has a nucleotide sequence of from about 13 to about 35 nucleotides selected from the nucleotide sequence of SEQ ID NO: 1. In still yet another embodiment thereof, the antisense oligonucleotide has a nucleotide sequence of from about 15 to about 26 nucleotides selected from the nucleotide sequence of SEQ ID NO: 1. In another embodiment thereof, the antisense oligonucleotide has a nucleotide sequence of from about 20 to about 26 nucleotides selected from the nucleotide sequence of SEQ ID NO: 1. In another embodiment thereof, the animal is administered an HDAC-7 antisense oligonucleotide having a nucleotide sequence of from about 13 to about 35 nucleotides and which comprises the nucleotide sequence of SEQ ID NO: 3. In another embodiment thereof, the animal is administered an HDAC-8 antisense oligonucleotide having a nucleotide sequence of from about 15 to about 26 nucleotides and which comprises the nucleotide sequence of SEQ ID NO: 3. In another embodiment thereof, the animal is administered an HDAC-8 antisense oligonucleotide that is SEQ ID NO: 4. In another embodiment thereof, the animal is a human. In another embodiment thereof, the method further comprises administering to an animal a therapeutically effective amount of an antisense oligonucleotide complementary to a region of RNA that encodes a portion of HDAC-1 or double-stranded DNA that encodes a portion of HDAC-1. In an embodiment thereof, the animal is administered a chimeric HDAC-1 antisense oligonucleotide. In another embodiment thereof, the animal is administered a hybrid HDAC-1 antisense oligonucleotide. In another embodiment thereof, the antisense oligonucleotide has a nucleotide sequence of from about 13 to about 35 nucleotides selected from the nucleotide sequence of SEQ ID NO: 5. In still yet another embodiment thereof, the antisense oligonucleotide has a nucleotide sequence of from about 15 to about 26 nucleotides selected from the nucleotide sequence of SEQ ID NO: 5. In another embodiment thereof, the antisense oligonucleotide has a nucleotide sequence of from about 20 to about 26 nucleotides selected from the nucleotide sequence of SEQ ID NO: 5. In another embodiment thereof, the animal is administered an HDAC-1 antisense oligonucleotide having a nucleotide sequence of from about 13 to about 35 nucleotides and which comprises the nucleotide sequence of SEQ ID NO: 5. In another embodiment thereof, the animal is administered an HDAC-1 antisense oligonucleotide having a nucleotide sequence of from about 15 to about 26 nucleotides and which comprises the nucleotide sequence of SEQ ID NO: 6. In yet another embodiment thereof, the animal is administered an HDAC-1 antisense oligonucleotide that is SEQ ID NO: 6.

In fourth aspect, the invention provides a method for inhibiting HDAC-7 and/or HDAC-8 activity in a cell, comprising contacting the cell with a small molecule inhibitor of HDAC-7 and/or HDAC-8, wherein HDAC-8 activity is inhibited.

In another embodiment therein, the invention provides a method wherein the inhibition of HDAC-7 and/or HDAC-8 activity in the contacted cell further leads to an inhibition of cell proliferation in the contacted cell. In another embodiment therein, the invention provides a method wherein inhibition of HDAC-7 and/or HDAC-8 activity in the contacted cell further leads to growth retardation of the contacted cell. In another embodiment therein, the invention provides a method wherein inhibition of HDAC-7 and/or HDAC-8 activity in the contacted cell further leads to growth arrest of the contacted cell. In another embodiment therein, the invention provides a method wherein inhibition of HDAC-7 and/or HDAC-8 activity in the contacted cell further leads to programmed cell death of the contacted cell. In another embodiment therein, the invention provides a method wherein inhibition of HDAC-7 and/or HDAC-8 activity in the contacted cell further leads to necrotic cell death of the contacted cell. In another embodiment thereof, the contacted cell is a human cell.

In fifth aspect, the invention provides a method for inhibiting neoplastic cell proliferation in an animal, comprising administering to an animal having at least one neoplastic cell present in its body a therapeutically effective amount of a small molecule inhibitor of HDAC-7 and/or HDAC-8, whereby neoplastic cell proliferation is inhibited.

In another embodiment thereof, the invention provides a method wherein the animal administered a small molecule inhibitor is a human.

In a sixth aspect, the invention provides a method for inhibiting the induction of cell proliferation, comprising contacting a cell with an antisense oligonucleotide that inhibits the expression of HDAC-7 or HDAC-8 and/or contacting a cell with a small molecule inhibitor of HDAC-7 and/or HDAC-8. In certain preferred embodiments, the cell is a neoplastic cell, and the induction of cell proliferation is tumorigenesis.

In a seventh aspect, the invention provides a method for identifying a small molecule histone deacetylase inhibitor that inhibits the HDAC-7 and/or HDAC-8 isoform, the isoform being required for the induction of cell proliferation. The method comprises contacting the HDAC-7 or HDAC-8 isoform with a candidate small molecule inhibitor and measuring the enzymatic activity of the contacted histone deacetylase isoform, wherein a reduction in the enzymatic activity of the contacted HDAC-7 or HDAC-8 isoform identifies the candidate small molecule inhibitor as a small molecule histone deacetylase inhibitor of the HDAC-7 or HDAC-8 isoform.

In an eighth aspect, the invention provides a method for identifying a small molecule histone deacetylase inhibitor that inhibits HDAC-7 or HDAC-8 isoform, which is involved in the induction of cell proliferation. The method comprises contacting a cell with a candidate small molecule inhibitor and measuring the enzymatic activity of the contacted histone deacetylase isoform, wherein a reduction in the enzymatic activity of the HDAC-7 or HDAC-8 isoform identifies the candidate small molecule inhibitor as a small molecule histone deacetylase inhibitor of HDAC-7 or HDAC-8.

In a ninth aspect, the invention provides a small molecule histone deacetylase inhibitor identified by the method of the seventh or the eighth aspect of the invention. Preferably, the histone deacetylase small molecule inhibitor is substantially pure.

In a tenth aspect, the invention provides a method for inhibiting cell proliferation in a cell comprising, contacting a cell with at least two reagents selected from the group consisting of an antisense oligonucleotide that inhibits expression of HDAC-7 or HDAC-8 isoform, a small molecule histone deacetylase inhibitor that inhibits expression or activity of HDAC-7 and/or HDAC-8 isoform, an antisense oligonucleotide that inhibits expression of the HDAC-1 isoform, a small molecule histone deacetylase inhibitor that inhibits the expression or the activity of the HDAC-1 isoform, an antisense oligonucleotide that inhibits expression of a DNA methyltransferase, and a small molecule DNA methyltransferase inhibitor. In certain embodiments, the inhibition of cell growth of the contacted cell is greater than the inhibition of cell growth of a cell contacted with one or more of the anti-HDAC-7 or anti-HDAC-8 reagents. In certain embodiments, each of the reagents selected from the group is substantially pure. In preferred embodiments, the cell is a neoplastic cell. In yet additional embodiments, the reagents selected from the group are operably associated.

In an eleventh aspect, the invention provides a method of inhibiting neoplastic cell growth, comprising contacting a cell with at least two reagents selected from the group consisting of an antisense oligonucleotide that inhibits expression of HDAC-7 or HDAC-8 isoform, a small molecule histone deacetylase inhibitor that inhibits the expression or the activity of HDAC-7 and/or HDAC-8 isoform, an antisense oligonucleotide that inhibits expression of the HDAC-1 isoform, a small molecule histone deacetylase inhibitor that inhibits expression or activity of the HDAC-1 isoform, an antisense oligonucleotide that inhibits expression of a DNA methyltransferase, and a small molecule DNA methyltransferase inhibitor. In some embodiments, the inhibition of cell growth of the contacted cell is greater than the inhibition of cell growth of a cell contacted with only one of the reagents. In certain embodiments, each of the reagents selected from the group is substantially pure. In preferred embodiments, the cell is a neoplastic cell. In yet additional preferred embodiments, the reagents selected from the group are operably associated.

### BRIEF DESCRIPTION OF THE DRAWINGS

FIG. 1 shows that expression of HDAC-7 mRNA was inhibited in a dose-dependent manner by both AS-1 and AS-2 oligos directed against human HDAC7 in human cancer A549 cells.

FIG. 2 shows that expression of HDAC-7 protein was inhibited and expression of p21 protein induced by both AS-1 and AS-2 oligos directed against human HDAC7 in human cancer A549 cells.

FIG. 3 shows time course analysis of expression of HDAC-7 mRNA by AS-1 oligo directed against human HDAC7 in human cancer A549 cells.

FIG. 4 shows time course analysis of HDAC-7 protein expression by both AS-1 and AS-2 oligos directed against human HDAC7 in human cancer A549 cells.

FIG. 5 shows that expression of HDAC-8 mRNA was inhibited in a dose-dependent manner by both AS-1 and AS-2 oligos directed against human HDAC8 in human cancer A549 cells.

FIG. 6 shows time course analysis of expression of HDAC-8 mRNA by AS-2 oligo directed against human HDAC8 in human cancer A549 cells.

FIG. 7 shows a growth curve of human cancer A549 cells treated with AS directed against human HDAC-7 (AS-1) or directed against human HDAC1 (AS-1).

FIG. 8 shows a growth curve of human cancer A549 cells treated with varying dose of human AS-1 or AS-2 oligos directed against human HDAC-8.

FIG. 9 shows cell cycle analysis of human A549 cancer cells treated with AS-1, AS-2 or MM-1 oligos directed against human HDAC7.

FIG. 10 shows cell cycle analysis of human A549 cancer cells treated with human HDAC8 antisense inhibitors and oxamflatin.

FIG. 11 shows dose-dependent induction of apoptosis of human cancer A549 cells by HDAC-8 and HDAC-1 antisense inhibitors.

FIG. 12 shows that HDAC-1 or HDAC-8 antisense inhibitor did not induce apoptosis in human normal epithelial HMEC cells.

FIG. 13 shows that similar inhibition of HDAC1 expression at the mRNA level by its antisense inhibitor leads to apoptosis of human cancer A549 cells but not normal HMEC cells.

FIG. 14 shows induction of apoptosis of human cancer A549 and T24 cells by HDAC-8 and HDAC-1 antisense inhibitors.

FIG. 15 shows time-dependence of apoptosis induction of human cancer A549 cells by HDAC-1 or HDAC-8 antisense or mismatch oligos.

FIG. 16 shows co-inhibition of HDAC-1 with HDAC-8, or HDAC-1 with HDAC-7, but not the other combinations, by antisense inhibitors synergized in induction of apoptosis of human cancer A549 cells.

FIG. 17 shows the nucleotide and amino acid sequences for HDAC-9, HDAC-10 and HDAC-11.

### DETAILED DESCRIPTION OF THE PREFERRED EMBODIMENTS

The patent and scientific literature referred to herein establishes knowledge that is available to those with skill in the art. The issued patents, applications, and references, including GenBank database sequences, that are cited herein are hereby incorporated by reference to the same extent as if each was specifically and individually indicated to be incorporated by reference.

The invention provides methods and reagents for modulating histone deacetylase (HDAC) isoforms, particularly HDAC-7 and HDAC-8, by inhibiting expression at the nucleic acid level or by inhibiting enzymatic activity at the protein level. The invention provides for the specific inhibition of specific histone deacetylase isoforms involved in tumorigenesis, and thus provides a treatment for cancer. The invention further provides for the specific inhibition of specific HDAC isoforms involved in cell proliferation and thus provides a treatment for cell proliferative disorders.

The inventors have made the surprising discovery that the specific inhibition of HDAC-7 and 8 dramatically induces apoptosis and growth arrest in cancerous cells. Accordingly, in a first aspect, the invention provides agents that inhibit the activity of the HDAC-7 and HDAC-8 isoforms.

In certain preferred embodiments of the first aspect of the invention, the agent that inhibits the HDAC-7 and HDAC-8 isoforms is an oligonucleotide that inhibits expression of a nucleic acid molecule encoding the HDAC-7 and HDAC-8 isoforms. The nucleic acid molecule encoding the HDAC-7 and HDAC-8 isoforms may be genomic DNA (e.g., a gene), cDNA, or RNA. In some embodiments, the oligonucleotide inhibits transcription of mRNA encoding the HDAC-7 or HDAC-8 isoforms. In other embodiments, the oligonucleotide inhibits translation of the HDAC-7 or HDAC-8 isoforms. In certain embodiments the oligonucleotide causes the degradation of the nucleic acid molecule.

In a preferred embodiment thereof, the agent of the first aspect of the invention is an antisense oligonucleotide complementary to a region of RNA that encodes a portion of HDAC-7 or HDAC-8 or to a region of double-stranded DNA that encodes a portion of HDAC-7 or HDAC-8 isoforms. In one embodiment thereof, the antisense oligonucleotide is a chimeric oligonucleotide. In another embodiment thereof, the antisense oligonucleotide is a hybrid oligonucleotide. In another embodiment thereof, the antisense oligonucleotide has a nucleotide sequence of from about 13 to about 35 nucleotides selected from the nucleotide sequence of SEQ ID NO: 1. In still yet another embodiment thereof, the antisense oligonucleotide has a nucleotide sequence of from about 15 to about 26 nucleotides selected from the nucleotide sequence of SEQ ID NO: 1. In another embodiment thereof, the antisense oligonucleotide has a nucleotide sequence of from about 20 to about 26 nucleotides selected from the nucleotide sequence of SEQ ID NO: 1. In another embodiment thereof, the antisense oligonucleotide has a nucleotide sequence of from about 13 to about 35 nucleotides and which comprises the nucleotide sequence of SEQ ID NO: 2. In still yet another embodiment thereof, the antisense oligonucleotide has a nucleotide sequence of from about 15 to about 26 nucleotides and which comprises the nucleotide sequence of SEQ ID NO: 2. In another embodiment thereof, the antisense oligonucleotide has a nucleotide sequence of from about 20 to about 26 nucleotides and which comprises the nucleotide sequence of SEQ ID NO: 2. In another embodiment thereof, the antisense oligonucleotide is SEQ ID NO: 3. In another embodiment thereof, the antisense oligonucleotide has one or more phosphorothioate internucleoside linkages. In another embodiment thereof, the antisense oligonucleotide further comprises a length of 20-26 nucleotides. In still another embodiment thereof, the antisense oligonucleotide is modified such that the terminal four nucleotides at the 5′ end of the oligonucleotide and the terminal four nucleotides at the 3′ end of the oligonucleotide each have 2′-O-methyl groups attached to their sugar residues.

In certain preferred embodiments of the first aspect, the agent that inhibits the HDAC-7 and/or HDAC-8 isoform in a cell is a small molecule inhibitor that inhibits expression of a nucleic acid molecule encoding HDAC-7 or HDAC-8 isoform or activity of the HDAC-7 and/or HDAC-8 protein.

The term “small molecule” as used in reference to the inhibition of histone deacetylase is used to identify a compound having a molecular weight preferably less than 1000 Da, more preferably less than 800 Da, and most preferably less than 600 Da, which is capable of interacting with a histone deacetylase and inhibiting the expression of a nucleic acid molecule encoding an HDAC isoform or activity of an HDAC protein. Inhibiting histone deacetylase enzymatic activity means reducing the ability of a histone deacetylase to remove an acetyl group from a histone. In some preferred embodiments, such reduction of histone deacetylase activity is at least about 50%, more preferably at least about 75%, and still more preferably at least about 90%. In other preferred embodiments, histone deacetylase activity is reduced by at least 95% and more preferably by at least 99%. In a particularly preferred embodiment, the small molecule inhibitor of HDAC is an inhibitor of HDAC-7 and/or HDAC-8.

Preferably, such inhibition is specific, i.e., the histone deacetylase inhibitor reduces the ability of a histone deacetylase to remove an acetyl group from a histone at a concentration that is lower than the concentration of the inhibitor that is required to produce another, unrelated biological effect. Preferably, the concentration of the inhibitor required for histone deacetylase inhibitory activity is at least 2-fold lower, more preferably at least 5-fold lower, even more preferably at least 10-fold lower, and most preferably at least 20-fold lower than the concentration required to produce an unrelated biological effect.

Preferred agents that inhibit HDAC-7 and/or HDAC-8 inhibit growth of human cancer cells, independent of their p53 status. These agents induce apoptosis in cancer cells and cause growth arrest. They also can induce transcription of p21^{WAF1} (a tumor suppressor gene), Bax, an extremely important gene involved in apoptosis regulation and GADD45, a stress-induced gene and important regulator of cell growth. These agents may exhibit both in vitro and in vivo anti-tumor activity. Inhibitory agents that achieve one or more of these results are considered within the scope of this aspect of the invention.

The antisense oligonucleotides according to the invention are complementary to a region of RNA or to a region of double-stranded DNA that encodes a portion of one or more histone deacetylase isoforms (taking into account that homology between different isoforms may allow a single antisense oligonucleotide to be complementary to a portion of more than one isoform). For purposes of the invention, the term “oligonucleotide” includes polymers of two or more deoxyribonucleosides, ribonucleosides, or any combination thereof. Preferably, such oligonucleotides have from about 6 to about 50 nucleoside residues, and most preferably from about 12 to about 30 nucleoside residues. The nucleoside residues may be coupled to each other by any of the numerous known internucleoside linkages. Such internucleoside linkages include without limitation phosphorothioate, phosphorodithioate, alkylphosphonate, alkylphosphonothioate, phosphotriester, phosphoramidate, siloxane, carbonate, carboxymethylester, acetamidate, carbamate, thioether, bridged phosphoramidate, bridged methylene phosphonate, bridged phosphorothioate, and sulfone internucleotide linkages. These internucleoside linkages preferably are phosphotriester, phosphorothioate, or phosphoramidate linkages, or combinations thereof.

Preferably, the oligonucleotides may also contain 2′-O-substituted ribonucleotides. For purposes of the invention the term “2′-O-substituted” means substitution of the 2′ position of the pentose moiety with an —O-lower alkyl group containing 1-6 saturated or unsaturated carbon atoms, or with an —O-aryl or allyl group having 2-6 carbon atoms, wherein such alkyl, aryl, or allyl group may be unsubstituted or may be substituted, e.g., with halo, hydroxy, trifluoromethyl, cyano, nitro, acyl, acyloxy, alkoxy, carboxyl, carbalkoxyl, or amino groups; or such 2′ substitution may be with a hydroxy group (to produce a ribonucleoside), an amino or a halo group, but not with a 2′-H group. The term “alkyl” as employed herein refers to straight and branched chain aliphatic groups having from 1 to 12 carbon atoms, preferably 1-8 carbon atoms, and more preferably 1-6 carbon atoms, which may be optionally substituted with one, two or three substituents. Unless otherwise apparent from context, the term “alkyl” is meant to include saturated, unsaturated, and partially unsaturated aliphatic groups. When unsaturated groups are particularly intended, the terms “alkenyl” or “alkynyl” will be used. When only saturated groups are intended, the term “saturated alkyl” will be used. Preferred saturated alkyl groups include, without limitation, methyl, ethyl, propyl, isopropyl, butyl, isobutyl, sec-butyl, tert-butyl, pentyl, and hexyl.

The term oligonucleotide also encompasses such polymers having chemically modified bases or sugars and/or having additional substituents including, without limitation, lipophilic groups, intercalating agents, diamines, and adamantane. The term oligonucleotide also encompasses such polymers as PNA and LNA.

For purposes of the invention, the term “complementary” means having the ability to hybridize to a genomic region, a gene, or an RNA transcript thereof, under physiological conditions. Such hybridization is ordinarily the result of base-specific hydrogen bonding between complementary strands, preferably to form Watson-Crick or Hoogsteen base pairs, although other modes of hydrogen bonding, as well as base stacking can lead to hybridization. As a practical matter, such hybridization can be inferred from the observation of specific gene expression inhibition, which may be at the level of transcription or translation (or both).

Particularly preferred antisense oligonucleotides utilized in this aspect of the invention include chimeric oligonucleotides and hybrid oligonucleotides.

For purposes of the invention, a “chimeric oligonucleotide” refers to an oligonucleotide having more than one type of internucleoside linkage. One preferred embodiment of such a chimeric oligonucleotide is a chimeric oligonucleotide comprising internucleoside linkages, phosphorothioate, phosphorodithioate, internucleoside linkages and phosphodiester, preferably comprising from about 2 to about 12 nucleotides. Some useful oligonucleotides of the invention have an alkylphosphonate-linked region and an alkylphosphonothioate region (see e.g., Pederson et al. U.S. Pat. Nos. 5,635,377 and 5,366,878). Preferably, such chimeric oligonucleotides contain at least three consecutive internucleoside linkages that are phosphodiester and phosphorothioate linkages, or combinations thereof.

For purposes of the invention, a “hybrid oligonucleotide” refers to an oligonucleotide having more than one type of nucleoside. One preferred embodiment of such a hybrid oligonucleotide comprises a ribonucleotide or 2′-O-substituted ribonucleotide region, preferably comprising from about 2 to about 12 2′-O-substituted nucleotides, and a deoxyribonucleotide region. Preferably, such a hybrid oligonucleotide contains at least three consecutive deoxyribonucleosides and contains ribonucleosides, 2′-O-substituted ribonucleosides, or combinations thereof (see e.g., Metelev and Agrawal, U.S. Pat. Nos. 5,652,355 and 5,652,356).

The exact nucleotide sequence and chemical structure of an antisense oligonucleotide utilized in the invention can be varied, so long as the oligonucleotide retains its ability to modulate expression of the target sequence, e.g., the HDAC-7 or the HDAC-8 isoform. This is readily determined by testing whether the particular antisense oligonucleotide is active by quantitating the amount of mRNA encoding the HDAC-7 or the HDAC-8 isoform, quantitating the amount of the HDAC-7 or the HDAC-8 isoform protein, quantitating the the HDAC-7 or the HDAC-8 isoform enzymatic activity, or quantitating the ability of the the HDAC-7 or the HDAC-8 isoform, for example, to inhibit cell growth in a an in vitro or in vivo cell growth assay, all of which are described in detail in this specification. The term “inhibit expression” and similar terms used herein are intended to encompass any one or more of these parameters.

Antisense oligonucleotides according to the invention may conveniently be synthesized on a suitable solid support using well-known chemical approaches, including H-phosphonate chemistry, phosphoramidite chemistry, or a combination of H-phosphonate chemistry and phosphoramidite chemistry (i.e., H-phosphonate chemistry for some cycles and phosphoramidite chemistry for other cycles). Suitable solid supports include any of the standard solid supports used for solid phase oligonucleotide synthesis, such as controlled-pore glass (CPG) (see, e.g., Pon, R. T., Meth. Molec. Biol. 20:465-496, 1993).

Antisense oligonucleotides according to the invention are useful for a variety of purposes. For example, they can be used as “probes” of the physiological function of specific histone deacetylase isoforms by being used to inhibit the activity of specific histone deacetylase isoforms in an experimental cell culture or animal system and to evaluate the effect of inhibiting such specific histone deacetylase isoform activity. This is accomplished by administering to a cell or an animal an antisense oligonucleotide that inhibits one or more histone deacetylase isoform expression according to the invention and observing any phenotypic effects. In this use, the antisense oligonucleotides used according to the invention are preferable to traditional “gene knockout” approaches because they are easier to use, and because they can be used to inhibit specific histone deacetylase isoform activity at selected stages of development or differentiation.

Preferred antisense oligonucleotides of the invention inhibit either the transcription of a nucleic acid molecule encoding the the HDAC-7 or the HDAC-8 isoform, and/or the translation of a nucleic acid molecule encoding the the HDAC-7 or the HDAC-8, and/or lead to the degradation of such nucleic acid molecules. HDAC-7- or HDAC-8-encoding nucleic acid molecules may be RNA or double stranded DNA regions and include, without limitation, intronic sequences, untranslated 5′ and 3′ regions, intron-exon boundaries, as well as coding sequences from the HDAC-7 or the HDAC-8 isoform genes.

Antisense oligonucleotides for human HDAC isotype polynucleotides may be designed from known HDAC isotype sequence data. For example, the following amino acid sequences are available from GenBank for HDAC-7, and HDAC-8: AAF63491, and AAF73076, respectively, and the following nucleotide sequences are available from GenBank for HDAC-7, and HDAC-8: AF239243, and AF230097, respectively.

The sequences encoding histone deacetylases from many non-human animal species are also known. Accordingly, the antisense oligonucleotides of the invention may also be complementary to a region of RNA or to a region of double-stranded DNA that encode the HDAC-7 or the HDAC-8 isoform from non-human animals. Antisense oligonucleotides according to these embodiments are useful as tools in animal models for studying the role of specific histone deacetylase isoforms.

Particularly, preferred oligonucleotides have nucleotide sequences of from about 13 to about 35 nucleotides which include the nucleotide sequences shown in Table 2 below.

These oligonucleotides have nucleotide sequences of from about 15 to about 26 nucleotides of the nucleotide sequences shown below in Table 2. Most preferably, the oligonucleotides shown below have phosphorothioate backbones, are 20-26 nucleotides in length, and are modified such that the terminal four nucleotides at the 5′ end of the oligonucleotide and the terminal four nucleotides at the 3′ end of the oligonucleotide each have 2′-O-methyl groups attached to their sugar residues.

The antisense oligonucleotides according to the invention may optionally be formulated with any of the well known pharmaceutically acceptable carriers or diluents (see preparation of pharmaceutically acceptable formulations in, e.g., Remington's Pharmaceutical Sciences, 18th Edition, ed. A. Gennaro, Mack Publishing Co., Easton, Pa., 1990), with the proviso that such carriers or diluents not affect their ability to modulate HDAC activity.

In certain preferred embodiments, the agent that inhibits the HDAC-7 and/or HDAC-8 isoform is a small molecule. In certain preferred embodiments, the small molecule inhibits the enzymatic activity of the HDAC-7 or HDAC-8 isoform.

Small molecule isotype-specific inhibitors of the invention may be conveniently prepared according to the following schemes or using other art-recognized methods.

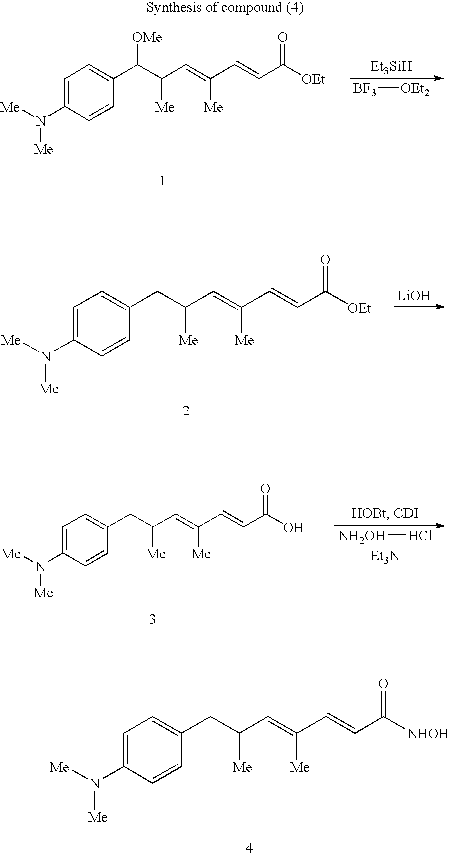

N-Hydroxy-4,6-dimethyl-7-[(4-N,N-dimethylaminophenyl)]-2,4-heptadienamide (4)

Step 1: Ethyl-4,6-dimethyl-7-[(4-N,N-dimethylaminophenyl)]-2,4-heptadienoate (2)

To a stirred solution of ester compound 1 (99 mg, 0.299 mmol) in CH₂Cl₂ (3 mL) at 0° C. was added triethylsilane (41.9 mg, 0.36 mmol) followed by BF₃.Et₂O (51 mg, 0.36 mmol) dropwise via microsyringe, and the mixture was stirred at 0° C. for 30 min. The reaction was quenched with saturated NaHCO₃ solution (3 mL), diluted with CH₂Cl₂ (20 mL) washed with water and the organic phase was dried and concentrated. Purification by flash silica gel chromatography (10% ethyl acetate in hexane) afforded the title compound 2 (87 mg, 97% yield) as a yellow oil.

¹H NMR (300 MHz, CDCl₃): δ7.29 (dd, J=15.6, 0.6 Hz, 1H), 6.98 (d, J=8.7 Hz, 2H), 6.64 (d, J=8.7 Hz, 2H), 5.74 (d, J=15.6 Hz, 1H), 5.73 (br d, J=10.2 Hz, 1H), 4.20 (q, J=6.9 Hz, 2H), 2.90 (s, 6H), 2.73 (m, 1H), 2.53 (d, J=7.2 Hz, 2H), 1.61 (d, J=0.6 Hz, 3H), 1.29 (t, J=6.9 Hz, 3H), 1.00 (d, J=6.6 Hz, 3H).

¹³C NMR (75 MHz, CDCl₃): δ12.1, 14.3, 20.0, 35.5, 40.8, 42.2, 60.1, 112.7, 115.5, 128.1, 129.7, 131.6, 147.5, 149.0, 149.8, 167.5.

Step 2: 4,6-Dimethyl-7-[(4-N,N-dimethylaminophenyl)]-2,4-heptadienoic acid (3)

To a stirred solution of diene ester 2 in methanol at r.t. was added aqueous LiOH 0.5 N solution. After being stirred at 40° C. for 16 hr., methanol was removed under reduced pressure and the resulting aqueous solution was acidified with HCl 3N (pH=ca. 4) extracted with ethyl acetate dried (MgSO₄), and concentrated under reduced pressure to give the desired carboxylic acid 3 as a yellow oil in 98% yield.

¹H NMR (300 MHz, CDCl₃): δ7.38 (dd, J=15.6, 0.6 Hz, 1H), 6.98 (d, J=9.0 Hz, 2H), 6.67 (d, J=9.0 Hz, 2H), 5.79 (br d, J=9.6 Hz, 1H), 5.73 (d, J=15.6 Hz, 1H), 2.91 (s, 6H), 2.76 (m, 1H), 2.57 (d, J=7.2 Hz, 2H), 1.62 (d, J=0.6 Hz, 3H), 1.01 (d, J=6.6 Hz, 3H).

¹³C NMR (75 MHz, CDCl₃):

δ12.2, 20.0,35.7,40.9,42.17, 112.9, 114.7, 128.2, 129.7, 131.7, 148.9, 149.1, 152.1, 172.7.

Step 3: N-Hydroxy-4,6-dimethyl-7-[(4-N,N-dimethylaminophenyl)]-2,4-heptadienamide (4)

To a stirred solution of carboxylic acid 3 (70 mg, 0.256 mmol) at r.t. in anhydrous DMF (2 mL) was added 1-hydroxybenzotriazole hydrate (41.5 mg, 0.307 mmol) followed by 1-(3-dimethylaminopropyl)-3-ethyl-carbodiimide hydrochloride(65 mg, 0.340 mmol). After 1 hr. hydroxylamine hydrochloride (89 mg, 1.28 mmol) and Et₃N (0.27 mL, 1.92 mmol) was added and stirring continued at r.t. overnight. The solvent was removed in vacuo and the residue obtained was diluted with ethylacetate (30 mL), washed with water and then saturated NaHCO₃ solution (5 mL). After drying and concentration, the crude product was purified by flash silica gel chromatography (2%-10% methanol in chloroform) to give the title compound 4 (30 mg, 41% yield) as a yellow oil.

¹H NMR (300 MHz, CDCl₃/CD₃OD=5/1): δ7.10 (d, J=14.4 Hz, 1H), 6.88 (d, J=8.7 Hz, 2H), 6.58 (d, J=8.7 Hz, 2H), 5.59 (d, J=9.3 Hz, 1H), 5.55 (br d, J=14.4 Hz, 1H), 2.78 (s, 6H), 2.63 (m, 1H), 2.40 (d, J=6.9 Hz, 2H), 1.48 (s, 3H), 0.89 (d, J=6.6 Hz, 3H).

¹³C NMR (75 MHz, CDCl₃/CD₃OD=5/1): δ11.8, 19.7, 35.3, 40.8, 42.0, 14.3, 20.0 35.5, 40.8, 42.2, 113.1, 113.7, 128.7, 129.5, 131.1, 145.9, 146.3, 148.9, 165.5.

Synthesis of Compound 5,

N-(2-Aminophenyl)-3-[4-(4-methylbenzenesulfonylamino)-phenyl]-acrylamide is described in Example 31 (compound 119) of WO 01/38322 which is hereby incorporated by reference.

Synthesis of Compound 13.

4-{[4-Amino-6-(2-indanyl-amino)-[1,3,5]-triazin-2-yl-amino]-methyl}-N-(2-amino-phenyl)-benzamide (13)

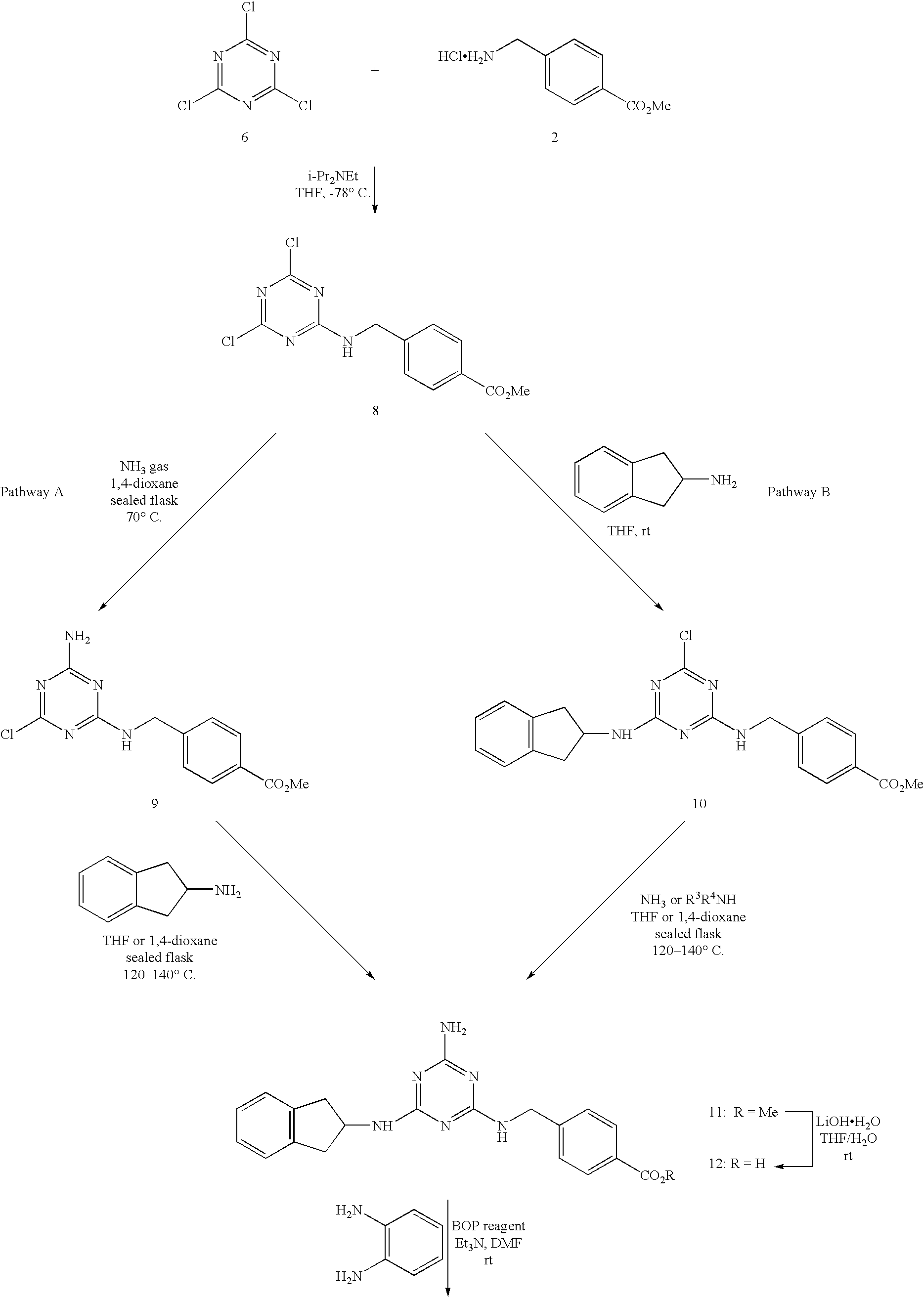

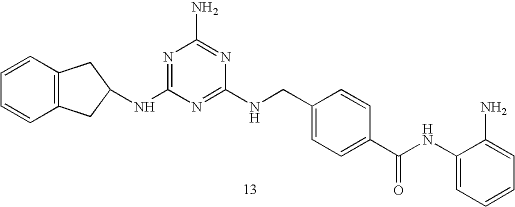

Step 1: Methyl-4-[(4,6-dichloro-[1,3,5]triazin-2-yl-amino)-methyl]-benzoate (8)

To a stirred solution at −78° C. of cyanuric chloride 6 (8.23 g, 44.63 mmol) in anhydrous THF (100 ml) under nitrogen was added a suspension of methyl 4-(aminomethyl)benzoate.HCl 7 (10.00 g, 49.59 mmol), in anhydrous THF (50 ml), followed by i-Pr₂NEt (19.00 ml, 109.10 mmol). After 30 min, the reaction mixture was poured into a saturated aqueous solution of NH₄Cl, and diluted with AcOEt. After separation, the organic layer was successively washed with sat. NH₄Cl, H₂O and brine, dried over anhydrous MgSO₄, filtered and concentrated. The crude residue was then purified by flash chromatography on silica gel (AcOEt/CH₂Cl₂: 5/95) to afford the title compound 8 (12.12 g, 38.70 mmol, 87% yield) as a pale yellow solid. ¹H NMR (300 MHz, CDCl₃) δ (ppm): AB system (δ_{A}=8.04, δ_{B}=7.38, J=8.5 Hz, 4H), 6.54 (bt, 1H), 4.76 (d, J=6.3 Hz, 2H), 3.93 (s, 3H).

Pathway A

Step 2: Methyl-4-[(4-amino-6-chloro-[1,3,5]triazin-2-yl-amino)-methyl]-benzoate (9)

In a 150 ml sealed flask, a solution of 8 (6.00 g, 19.16 mmol) in anhydrous 1,4-dioxane (60 ml) was stirred at room temperature, saturated with NH₃ gas for 5 min, and warmed to 70° C. for 6 h. The reaction mixture was allowed to cool to room temperature, the saturation step with NH₃ gas was repeated at room temperature for 5 min, and the reaction mixture was warmed to 70° C. again for 18 h. Then, the reaction mixture was allowed to cool to room temperature, poured into a saturated aqueous solution of NH₄Cl, and diluted with AcOEt. After separation, the organic layer was successively washed with sat. NH₄Cl, H₂O and brine, dried over anhydrous MgSO₄, filtered and concentrated. The crude residue was then purified by flash chromatography on silica gel (AcOEt/CH₂Cl₂: 30/70) to afford the title compound 9 (5.16 g, 17.57 mmol, 91% yield) as a white solid. ¹H NMR (300 MHz, CDCl₃) δ(ppm): AB system (δ_{A}=8.01, δ_{B}=7.35, J=8.1 Hz, 4H), 5.79 (bs, 1H), 5.40−5.20 (m, 2H), 4.72−4.63 (m, 2H), 3.91 (s, 3H).

Pathway B:

Step 2: Methyl 4-[(4-chloro-6-(2-indanyl-amino)-[1,3,5]triazin-2-yl-amino)-methyl]-benzoate (10)

To a stirred solution at room temperature of 8 (3.00 g, 9.58 mmol) in anhydrous THF (50 ml) under nitrogen were added i-Pr₂NEt (8.34 ml, 47.90 mmol) and 2-aminoindan.HCl (1.95 g, 11.50 mmol). After 18 h, the reaction mixture was poured into a saturated aqueous solution of NH₄Cl, and diluted with AcOEt. After separation, the organic layer was successively washed with sat. NH₄Cl, H₂O and brine, dried over anhydrous MgSO₄, filtered and concentrated to afford the title compound 10 (4.06 g, 9.91 mmol, quantitative yield) as a white powder. ¹H NMR (300 MHz, CDCl₃) δ(ppm): mixture of rotamers, 8.06−7.94 (m, 2H), 7.43−7.28 (m, 2H), 7.24−7.12 (m, 4H), 6.41 and 6.05 (2 bt, 1H), 5.68−5.44 (m, 1H), 4.92−4.54 (m, 3H), 3.92 (bs, 3H), 3.41−3.12 (m, 2H), 2.90−2.70 (m, 2H).

Step 3: Methyl-4-[(4-amino-6-(2-indanyl-amino)-[1,3,5]triazin-2-yl-amino)-methyl]-benzoate (11)

General Procedure for the Amination with NH₃ Gas:

In a 150 ml sealed flask, a solution of 10 (3.90 g, 9.51 mmol) in anhydrous 1,4-dioxane (80 ml) was stirred at room temperature, saturated with NH₃ gas for 5 min, and warmed to 140° C. for 6 h. The reaction mixture was allowed to cool to room temperature, the saturation step with NH₃ gas was repeated for 5 min, and the reaction mixture was warmed to 140° C. again for 18 h. Then, the reaction mixture was allowed to cool to room temperature, poured into a saturated aqueous solution of NH₄Cl, and diluted with AcOEt. After separation, the organic layer was successively washed with sat. NH₄Cl, H₂O and brine, dried over anhydrous MgSO₄, filtered and concentrated. The crude residue was then purified by flash chromatography on silica gel (MeOH/CH₂Cl₂: 3/97) to afford the title compound 11 (3.50 g, 8.96 mmol, 94% yield) as a pale yellow sticky solid. ¹H NMR (300 MHz, CDCl₃) δ(ppm): 7.99 (bd, J=8.2 Hz, 2H), 7.41−7.33 (m, 2H), 7.24−7.13 (m, 4H), 5.50−5.00 (m, 2H), 4.90−4.55 (m, 5H), 3.92 (s, 3H), 3.40−3.10 (m, 2H), 2.90−2.70 (m, 2H). ¹³C NMR: (75 MHz, CDCl₃) δ(ppm): 166.88, 167.35, 166.07, 144.77, 141.07, 129.82, 128.93, 127.01, 126.61, 124.70, 52.06, 51.80, 44.25, 40.16. HRMS (calc.) : 390.1804, (found): 390.1800.

Step 4:4-[(4-Amino-6-(2-indanyl-amino)-[1,3,5]triazin-2-yl-amino)-methyl]-benzoic acid (12)

To a stirred solution at room temperature of 11 (2.07 g, 5.30 mmol) in THF (50 ml) was added a solution of LiOH.H₂O (334 mg, 7.96 mmol) in water (25 ml). After 18 h, the reaction mixture was diluted in water and acidified with 1N HCl until pH 5-6 in order to get a white precipitate. After 1 h, the suspension was filtered off and the cake was abundantly washed with water, and dried to afford the title compound 12 (1.73 g, 4.60 mmol, 87% yield) as a white solid. ¹H NMR (300 MHz, acetone-d₆) δ(ppm): 8.05 (bd, J=8.1 Hz, 2H), 7.56−7.42 (m, 2H), 7.30−7.10 (m, 4H), 5.90−5.65 (m, 2H), 4.85−4.60 (m, 4H), 3.40−2.80 (m, 4H). HRMS (calc.): 376.1648, (found): 376.1651.

Step 5: 4-{[4-Amino-6-(2-indanyl-amino)-[1,3,5]-triazin-2-yl-amino]-methyl}-N-(2-amino-phenyl)-benzamide (13)

To a stirred solution at room temperature of 12 (200 mg, 0.53 mmol) in anhydrous DMF (5 ml) under nitrogen were added Et₃N (74 μl, 0.53 mmol) and BOP reagent (282 mg, 0.64 mmol), respectively. After 40 min, a solution of 1,2-phenylenediamine (64 mg, 0.58 mmol), Et₃N (222 μl, 1.59 mmol) in anhydrous DMF (2 ml) was added dropwise. After 1.5 h, the reaction mixture was poured into a saturated aqueous solution of NH₄Cl, and diluted with AcOEt. After separation, the organic layer was successively washed with sat. NH₄Cl, H₂O and brine, dried over anhydrous MgSO₄, filtered and concentrated. The crude residue was then purified by flash chromatography on silica gel (MeOH/CH₂Cl₂: 2/98→5/95) to afford the title compound 13 (155 mg, 0.33 mmol, 63% yield) as a pale yellow foam. ¹H NMR (300 MHz, acetone-d₆) δ(ppm): 9.04 (bs, 1H), 7.96 (bd, J=8.0 Hz, 2H), 7.50−7.40 (m, 2H), 7.30 (dd, J=8.0 Hz, 1.4 Hz, 1H), 7.22−7.08 (m, 4H), 6.99 (ddd, J=8.0 Hz, 7.5 Hz, 1.5 Hz, 1H), 6.86 (dd, J=8.0 Hz, 1.4 Hz, 1H), 6.67 (dt, J=7.5 Hz, 1.4 Hz, 1H), 6.60−5.49 (m, 4H), 4.80−4.50 (m, 4H), 3.30−3.08 (m, 2H), 2.96−2.74 (m, 2H).

Synthesis of Compound 16.

N-(2-Amino-phenyl)-4-(1H-benzimidazol-2-ylsulfanylmethyl)-benzamide (16)

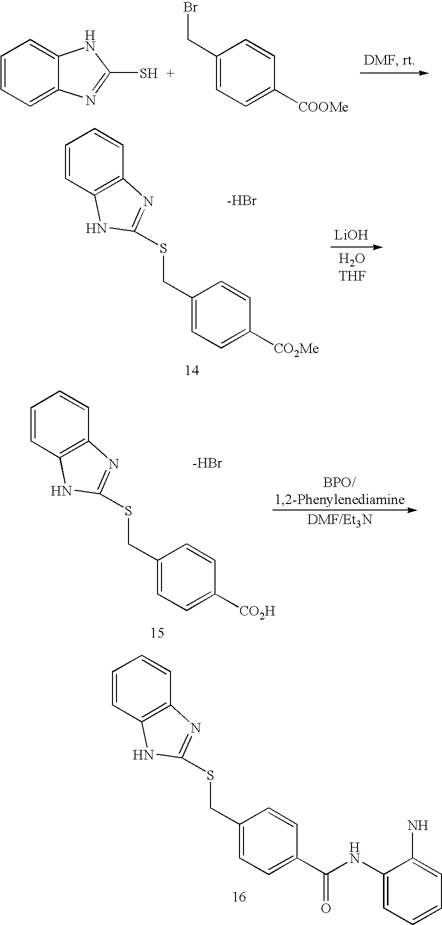

Step 1: 4-(1H-Benzimidazol-2-ylsulfanylmethyl)-benzoic acid methyl ester (14)

To a suspension of 2-mercaptobenzimidazole (2.0 g., 13.3 mmol) in DMF (66 mL) was added methyl 4-(bromomethyl)benzoate (3.0 g, 13.3 mmol). The mixture was stirred at r.t. for 1 hr. and evaporated to dryness. The resulting solid was dispersed in ether and collected by filtration affording the title compound 14 (4.03 g.) as a white solid. in 80% yield. LRMS=299.1 (M+1).

Step 2: N-(2-Amino-phenyl)-4-(1H-benzimidazol-2-ylsulfanylmethyl)-benzamide (16)

To a stirred solution at room temperature of 14 (4.19 g, 11.0 mmol) in THF (30 ml) was added a solution of LiOH.H₂O (1.58 g. 66.3 mmol) in water (30 ml). After 16 h, the reaction mixture was heated at 50° C. for 3 hr. diluted in water and acidified with 1N HCl until pH 4-5 in order to get a white precipitate. After 1 h, the solid was collected by filtration and thoroughly washed with water and dried to afford the corresponding acid 15 (3.0 g.) as a white solid in 96% yield. LRMS=285.0 (M+1)

To a stirred solution at room temperature of 15 (3.0 g, 10.6 mmol) in anhydrous DMF (50 ml) under nitrogen were added Et₃N (1.53 ml, 11.0 mmol) and a solution of BOP reagent (5.13 g, 11.6 mmol in 25 ml DMF) respectively. After 40 min, a solution of 1,2-phenylenediamine (1.26 g, in 25 ml DMF 11.6 mmol) was transferred via canula followed by Et₃N (4.4 ml, 31.7 mmol). After 1.5 h, DMF was removed in vaco at 80° C. and the resulting syrup was crystallized by adding AcOEt. The crystals were dissolved in a minimum amount of DMF and crystallized by adding hot AcOEt to afford the title compound 16 (1.71 g) in 43% yield.

¹H NMR: (DMSO-d₆) δ(ppm): 9.57 (s, 1H), 7.89 (d, J=8.2 Hz, 2H), 7.55 (d, J=8.2 Hz, 2H), 7.53 (bs, 2H), 7.36 (bs, 2H), 7.14−7.08 (m, 3H), 6.94 (t, J=8.2 Hz, 1H), 6.74 (d, J=6.9 Hz, 1H), 6.56 (t, J=8.0 Hz, 1H), 4.87 (bs, 2H), 4.62 (s, 2H).

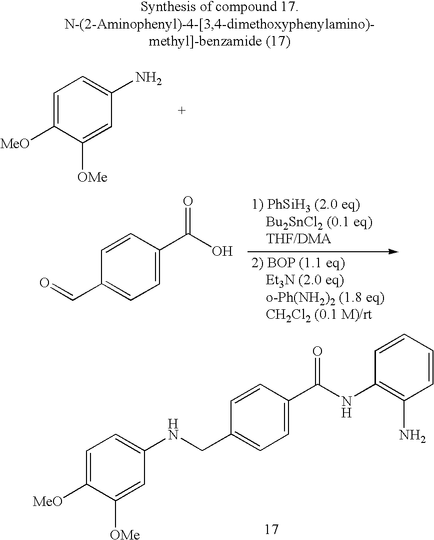

Step 1. 4-[(3,4-Dimethoxyphenylamino)-methyl]-benzoic acid

In a 50 ml flask, a mixture of 4-aminoveratrole (1.53 g, 10 mmol), 4-formyl-benzoic acid (1.50 g, 10 mmol), dibutyltin dichloride (304 mg, 1 mmol), phenylsilane (2.47 ml, 20 mmol) in anhydrous THF (10 m) and DMA (10 ml) was stirred at r.t. overnight. After solvents removal, the crude residue was dissolved in EtOAc (100 ml) and then washed with saturated aqueous solution of NaHCO₃ (50 ml×3). The combined aqueous layer was acidified with 6% of NaHSO₄ to pH=4. The resulting white suspension was filtrated and then the filter cake was washed with water (5 ml×3). The cake was dried over freeze dryer to afford acid (1.92 g, 67%) white solid product.

LRMS=288 (M+1).

Step 2. N-(2-Aminophenyl)-4-[(3,4-dimethoxyphenylamino)-methyl]-benzamide (17)

In a 150 ml flask, a mixture of acid (1.92 g, 6.69 mmol) from step 1, benzotriazol-1-yloxy-tris-(dimethylamino)-phosphonium hexafluorophosphate (BOP, 3.26 g, 7.37 mmol), triethylamine (1.87 ml, 13.4 mmol), o-phenylenediamine (1.30 g, 12.02 mmol) in methylenechloride (67 ml) was stirred at r.t. for 2 h. After solvents removal, the crude residue was dissolved in EtOAc (100 ml) and then washed with NaHCO₃ saturated solution and brine 50 ml. The combined organic layers were dried over Na₂SO₄ and the filtrate was concentrated to dryness. The crude material was submitted to a chromatographic purification (column silica, 55%-70% EtOAc in 1% Et₃N of hexanes) and then the all interested fractions were concentrated to dryness. The residue was suspended in minimum quantities of EtOAc and then filtered to afford final product 17 (1.49 g, 59%).

¹H NMR (300 MHz, DMSO-d₆) δ (ppm): 9.65 (s, 1H), 7.98 (d, J=7.9 Hz, 2H), 7.54 (d, J=7.9 Hz, 2H), 7.22 (d, J=7.9 Hz, 1H), 7.02 (dd, J=7.9 Hz, 7.9 Hz, 1H), 6.83 (d, J=7.9 Hz, 1H), 6.72 (d, J=8.79 Hz, 1H), 6.45 (dd, J=7.49 Hz, 7.49 Hz, 1H), 6.39 (d, J=2.2 Hz, 1H), 6.01-6.08 (m, 2H), 4.94 (s, 2H, NH₂), 4.36 (d, J=6.16 Hz, 2H), 3.72 (s, 3H), 3.65 (s, 3H).

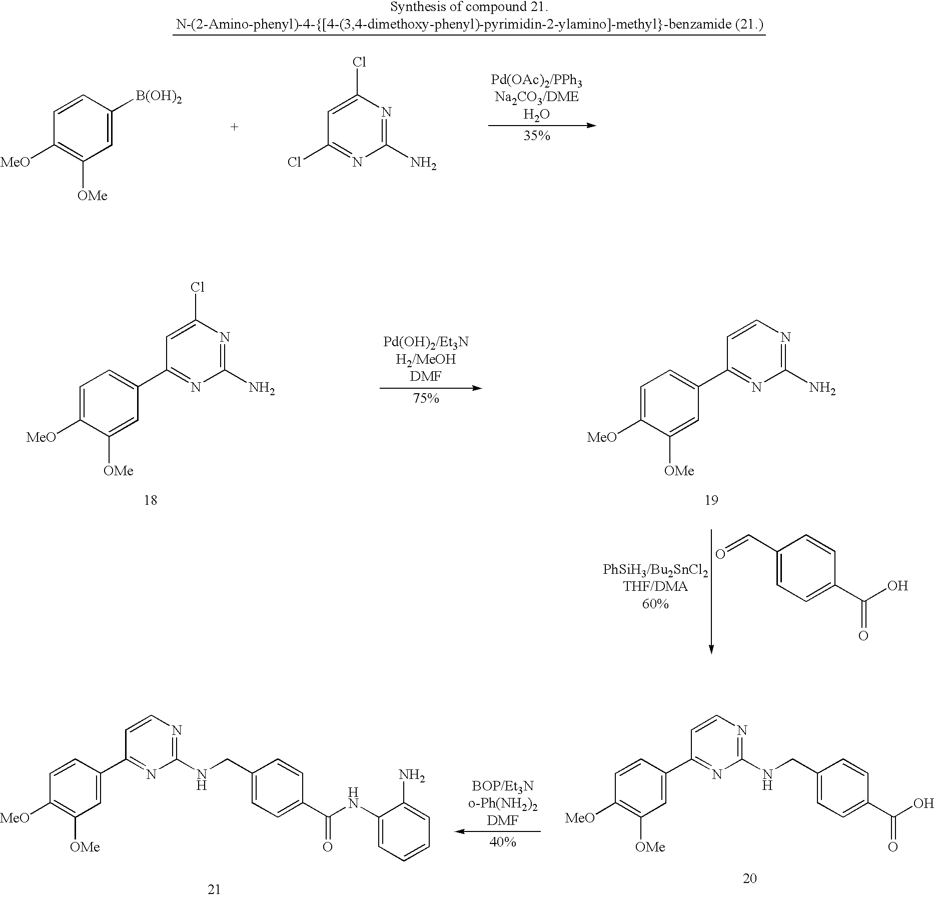

Step 1: 4-Chloro-6-(3,4-dimethoxy-phenyl)-pyrimidin-2-ylamine (18)

In a 250 ml flask, a mixture of 3,4-dimethoxybenzeneboronic acid (1.12 g, 6.15 mmol), 2-amino-4,6-dichloro-pyrimidine (2.0 g, 12.2 mmol), palladium diacetate (0.276 g, 1.22 mmol), and triphenylphosphine (0.648 g, 2.47 mmol) were suspended in anhydrous DME (120 ml) under N₂ atmosphere. A solution of Na₂CO₃ (4.06 g, 38 mmol) in minimum quantities of H₂O (18 ml) was added to the mixture. The reflux condenser was applied and the mixture was heated to reflux overnight. The reaction mixture was concentrated to dryness and then purified by flash chromatography (silica gel, 25%-35% EtOAc in 1% Et₃N of hexanes) to give compound 18 (0.64 g, 39%) as a pale yellow solid. LRMS 266 (M+1).

Step 2: 4-(3,4-Dimethoxy-phenyl)-pyrimidin-2-ylamine (19)

In a 50 ml flask, compound 18 (0.55 g, 2.07 mmol) was dissolved in a mixture of MeOH (10 ml) and DMF (10 ml) under N₂ atmosphere. Triethylamine (0.6 ml, 4.3 mmol) and palladium hydroxide (0.4 g, 20% wt. % Pd on carbon) were added in turn. A H₂ balloon was then applied and the mixture was stirred overnight at rt. The mixture was evaporated to dryness. The residue was dissolved in EtOAc (200 ml) and then washed with a saturated solution of NaHCO₃ (50 ml×2) and brine (50 ml). The organic layer was dried over Na₂SO₄, filtered and concentrated to dryness to give compound 19 (0.424 g, 75%) as a off-white solid. LRMS 232 (M+1).

4-{[4-(3,4-Dimethoxy-phenyl)-pyrimidin-2-ylamino]-methyl}-benzoic acid (20)

In a 50 ml flask, a mixture of compound 19 (0.424 g, 1.83 mmol), 4-formyl-benzoic acid (0.262 g, 1.74 mmol), dibutyl tin dichloride (35 mg, 0.174 mmol), phenyl silane (0.429 ml, 3.48 mmol) in anhydrous THF (1.83 ml) and DMA (1.83 ml) was stirred at r.t. overnight. After solvents removal, the crude residue was dissolved in EtOAc (100 ml) and then washed with saturated aqueous solution of NaHCO₃ (50 ml×3). The combined aqueous layer was acidified with 6% of NaHSO₄ to pH=3-4. The resulting white suspension was filtrated and then the filter cake was washed with water (5 ml×3). The cake was dried over freeze dryer to afford acid 20 (0.4 g, 60%) white solid product. LRMS=366 (M+1).

N-(2-Amino-phenyl)-4-{[4-(3,4-dimethoxy-phenyl)-pyrimidin-2-ylamino]-methyl}-benzamide (21)

In a 50 ml flask, a mixture of compound 20 (400 mg, 1.10 mmol), benzotriazol-1-yloxy-tris(dimethylamino)phosphonium hexafluorophosphate (BOP, 581 mg, 1.31 mmol), triethylamine (0.31 ml, 2.19 mmol), o-phenylenediamine (0.237 mg, 2.19 mmol) in anhydrous DMF (11 ml) was stirred at rt for 2 h. After solvents removal, the residue was dissolved in EtOAc (150 ml) and then washed with a saturated solution of NaHCO₃ (50 ml×3) and brine (50 ml). The combined organic layers were dried over Na₂SO₄ and the filtrate was concentrated to dryness. The crude material was recrystallized in EtOAc to give the title product 21 (200 mg, 40%) as a off-white solid.

¹H NMR (300 MHz, DMSO-d₆) δ (ppm): 9.64 (s, 1H), 8.35 (d, J=4.8 Hz, 7.97 (d, J=7.9 Hz, 2H), 7.89 (m, 1H), 7.72 (m, 2H), 7.55 (d, J=7.5 Hz, 2H), 7.2 (d, J=5.3 Hz, 2H), 7.10 (d, J=8.4 Hz, 1H), 7.01 (m, 1H), 6.82 (d, J=7.0 Hz, 1H), 6.41 (t, J=7.5 Hz, 1 H), 4.92 (s, 2H, NH₂), 4.68 (d, J=6.16 Hz, 2H), 3.82 (s, 6H)

The reagents according to the invention are useful as analytical tools and as therapeutic tools, including gene therapy tools. The invention also provides methods and compositions which may be manipulated and fine-tuned to fit the condition(s) to be treated while producing fewer side effects.

The invention also provides methods for inhibiting HDAC-7 and/or 8 activity in a cell, comprising contacting the cell with a specific inhibitor of HDAC-7 and/or 8, whereby HDAC-7 and/or HDAC-8 activity is inhibited. As used herein, the term “specific inhibitor” means any molecule or compound that decreases the amount of HDAC-7 or HDAC-8 RNA, HDAC-7 or HDAC-8 protein, and/or HDAC-7 or HDAC-8 protein activity in a cell, relative to other isoforms of HDAC. In an embodiment thereof, the invention provides a method for inhibiting the HDAC-7 or HDAC-8 isoform in a cell comprising contacting the cell with an antisense oligonucleotide of the first aspect of the invention. Preferably, cell proliferation is inhibited in the contacted cell. In preferred embodiments, the cell is a neoplastic cell which may be in an animal, including a human, and which may be in a neoplastic growth. In certain preferred embodiments, the method of the second aspect of the invention further comprises contacting the cell with a HDAC-7 and/or HDAC-8 small molecule inhibitor that interacts with and reduces the enzymatic activity of the HDAC-7 and/or 8 isoform. In some embodiments, the histone deacetylase small molecule inhibitor is operably associated with the antisense oligonucleotide.

Thus, the antisense oligonucleotides according to the invention are useful in therapeutic approaches to human diseases, including benign and malignant neoplasms, by inhibiting cell proliferation in cells contacted with the antisense oligonucleotides. The phrase “inhibiting cell proliferation” is used to denote an ability of an HDAC-7 or HDAC-8 antisense oligonucleotide or a small molecule HDAC-7 and/or HDAC-8 inhibitor (or combination thereof) to retard the growth of cells contacted with the oligonucleotide or small molecule inhibitor, as compared to cells not contacted.

An assessment of cell proliferation can be made by counting cells that have been contacted with the oligonucleotide or small molecule of the invention and compare that number with the number of non-contacted cells using a Coulter Cell Counter (Coulter, Miami, Fla.) or a hemacytometer. Where the cells are in a solid growth (e.g., a solid tumor or organ), such an assessment of cell proliferation can be made by measuring the growth of the tissue or organ with calipers, and comparing the size of the growth of contacted cells with non-contacted cells. Preferably, the term includes a retardation of cell proliferation that is at least 50% of non-contacted cells. More preferably, the term includes a retardation of cell proliferation that is 100% of non-contacted cells (i.e., the contacted cells do not increase in number or size). Most preferably, the term includes a reduction in the number or size of contacted cells, as compared to non-contacted cells. Thus, HDAC-7 or HDAC-8 antisense oligonucleotides or HDAC-7 and/or HDAC-8 small molecule inhibitors that inhibit cell proliferation in a contacted cell may induce the contacted cell to undergo growth retardation, growth arrest, programmed cell death (i.e., to apoptose), or necrotic cell death.

The anti-neoplastic utility of the antisense oligonucleotides according to the invention is described in detail elsewhere in this specification.

In yet other preferred embodiments, the cell contacted with HDAC-7 or HDAC-8 antisense oligonucleotide is also contacted with HDAC-7 and/or HDAC-8 small molecule inhibitor.

As used herein, the term “histone deacetylase small molecule inhibitor” denotes an active moiety capable of interacting with one or more specific histone deacetylase isoforms at the protein level and reducing the activity of that histone deacetylase isoform. Particularly preferred are histone deacteylase small molecule inhibitors that inhibit the HDAC-7 and/or the HDAC-8 isoform. An HDAC-1 small molecule inhibitor is a molecule that reduces the activity of the HDAC-1 isoform. An HDAC-7 small molecule inhibitor is a molecule that reduces the activity of the HDAC-7 isoform. An HDAC-8 small molecule inhibitor is a molecule that reduces the activity of the HDAC-8 isoform. In a preferred embodiment, the reduction of activity is at least 5-fold, more preferably at least 10-fold, most preferably at least 50-fold. In another embodiment, the activity of the histone deacetylase isoform is reduced 100-fold. As discussed below, a preferred histone deacetylase small molecule inhibitor is one that interacts with and reduces the enzymatic activity of HDAC-7 and/or the HDAC-8 isoform that is involved in tumorigenesis.

In a few preferred embodiments, the histone deacetylase small molecule inhibitor is operably associated with the antisense oligonucleotide. As mentioned above, the antisense oligonucleotides according to the invention may optionally be formulated well known pharmaceutically acceptable carriers or diluents. This formulation may further contain one or more one or more additional histone deacetylase antisense oligonucleotide(s), and/or one or more histone deacetylase small molecule inhibitor(s), or it may contain any other pharmacologically active agent.

The term “operably associated with” or “operable association” includes any association between the antisense oligonucleotide and the histone deacetylase small molecule inhibitor which allows an antisense oligonucleotide to inhibit one or more specific histone deacetylase isoform-encoding nucleic acid expression and allows the histone deacetylase small molecule inhibitor to inhibit specific histone deacetylase isoform enzymatic activity. One or more antisense oligonucleotide of the invention may be operably associated with one or more histone deacetylase small molecule inhibitor. In some preferred embodiments, an antisense oligonucleotide of the invention that targets one particular histone deacetylase isoform (e.g., HDAC-7 or HDAC-8) is operably associated with an small molecule inhibitor which targets the same histone deacetylase isoform. A preferred operable association is a hydrolyzable. Preferably, the hydrolyzable association is a covalent linkage between the antisense oligonucleotide and the histone deacetylase small molecule inhibitor. Such a covalent linkage is hydrolyzable, for example, by esterases and/or amidases. Examples of such hydrolyzable associations are well known in the art. Phosphate esters are particularly preferred.

In certain preferred embodiments, the covalent linkage may be directly between the antisense oligonucleotide and the histone deacetylase small molecule inhibitor so as to integrate the histone deacetylase small molecule inhibitor into the backbone of the oligonucleotide. Alternatively, the covalent linkage may be through an extended structure and may be formed by covalently linking the antisense oligonucleotide to the histone deacetylase small molecule inhibitor through coupling of both the antisense oligonucleotide and the histone deacetylase small molecule inhibitor to a carrier molecule such as a carbohydrate, a peptide, a lipid or a glycolipid. Another useful operable associations include lipophilic association, such as the formation of a liposome containing an antisense oligonucleotide and the histone deacetylase small molecule inhibitor covalently linked to a lipophilic molecule. Such lipophilic molecules include, without limitation, phosphotidylcholine, cholesterol, phosphatidylethanolamine, and synthetic neoglycolipids, such as syalyllacNAc-HDPE. In certain preferred embodiments, the operable association may not be a physical association, but simply a simultaneous co-existence in the body, for example, when the antisense oligonucleotide is associated with one liposome and the small molecule inhibitor is associated with another liposome.

In a third aspect, the invention provides a method for inhibiting neoplastic cell proliferation in an animal, comprising administering to an animal having at least one neoplastic cell present in its body a therapeutically effective amount of a specific inhibitor of HDAC-7 and/or 8, whereby neoplastic cell proliferation is inhibited in the animal. In an embodiment thereof, the invention provides a method for inhibiting neoplastic cell growth in an animal. In this method, a therapeutically effective amount of the antisense oligonucleotide of the invention is administered to an animal having at least one neoplastic cell present in its body, the oligonucleotide being administered with a pharmaceutically acceptable carrier for a therapeutically effective period of time. Preferably, the animal is a mammal, particularly a domesticated mammal. Most preferably, the animal is a human.

The term “neoplastic cell” is used to denote a cell that shows aberrant cell growth. A neoplastic cell may be a hyperplastic cell, a cell that shows a lack of contact inhibition of growth in vitro, a benign tumor cell that is incapable of metastasis in vivo, or a cancer cell that is capable of metastases in vivo and that may recur after attempted removal. The term “tumorigenesis” is used to denote the induction of uncharacteristic or untimely cell proliferation that leads to the development of a neoplastic growth.

As used herein, the term “therapeutically effective amount” means the total amount of each active component of the pharmaceutical composition or method that is sufficient to show a meaningful patient benefit, i.e., inhibiting HDAC activity, particularly HDAC-7 and/or HDAC-8 activity or to inhibit neoplastic growth or for the treatment of proliferative diseases and disorders. When applied to an individual active ingredient, administered alone, the term refers to that ingredient alone. When applied to a combination, the term refers to combined amounts of the active ingredients that result in the therapeutic effect, whether administered in combination, serially or simultaneously.

Administration of the synthetic oligonucleotide of the invention used in the pharmaceutical composition or to practice the method of the present invention can be carried out in a variety of conventional ways, such as intraocular, oral ingestion, inhalation, or cutaneous, subcutaneous, intramuscular, or intravenous injection.

When a therapeutically effective amount of synthetic oligonucleotide of the invention is administered orally, the synthetic oligonucleotide will be in the form of a tablet, capsule, powder, solution or elixir. When administered in tablet form, the pharmaceutical composition of the invention may additionally contain a solid carrier such as a gelatin or an adjuvant. The tablet, capsule, and powder contain from about 5 to 95% synthetic oligonucleotide and preferably from about 25 to 90% synthetic oligonucleotide. When administered in liquid form, a liquid carrier such as water, petroleum, oils of animal or plant origin such as peanut oil, mineral oil, soybean oil, sesame oil, or synthetic oils may be added. The liquid form of the pharmaceutical composition may further contain physiological saline solution, dextrose or other saccharide solution, or glycols such as ethylene glycol, propylene glycol or polyethylene glycol. When administered in liquid form, the pharmaceutical composition contains from about 0.5 to 90% by weight of the synthetic oligonucleotide and preferably from about 1 to 50% synthetic oligonucleotide.

When a therapeutically effective amount of synthetic oligonucleotide of the invention is administered by intravenous, subcutaneous, intramuscular, intraocular, or intraperitoneal injection, the synthetic oligonucleotide will be in the form of a pyrogen-free, parenterally acceptable aqueous solution. The preparation of such parenterally acceptable solutions, having due regard to pH, isotonicity, stability, and the like, is within the skill in the art. A preferred pharmaceutical composition for intravenous, subcutaneous, intramuscular, intraperitoneal, or intraocular injection should contain, in addition to the synthetic oligonucleotide, an isotonic vehicle such as Sodium Chloride Injection, Ringer's Injection, Dextrose Injection, Dextrose and Sodium Chloride Injection, Lactated Ringer's Injection, or other vehicle as known in the art. The pharmaceutical composition of the present invention may also contain stabilizers, preservatives, buffers, antioxidants, or other additives known to those of skill in the art.

The amount of synthetic oligonucleotide in the pharmaceutical composition of the present invention will depend upon the nature and severity of the condition being treated, and on the nature of prior treatments which the patent has undergone. Ultimately, the attending physician will decide the amount of synthetic oligonucleotide with which to treat each individual patient. Initially, the attending physician will administer low doses of the synthetic oligonucleotide and observe the patient's response. Larger doses of synthetic oligonucleotide may be administered until the optimal therapeutic effect is obtained for the patient, and at that point the dosage is not increased further. It is contemplated that the various pharmaceutical compositions used to practice the method of the present invention should contain about 10 μg to about 20 mg of synthetic oligonucleotide per kg body or organ weight.

The duration of intravenous therapy using the pharmaceutical composition of the present invention will vary, depending on the severity of the disease being treated and the condition and potential idiosyncratic response of each individual patient. Ultimately the attending physician will decide on the appropriate duration of intravenous therapy using the pharmaceutical composition of the present invention.

In a preferred embodiment, the therapeutic composition of the invention is administered systemically at a sufficient dosage to attain a blood level of antisense oligonucleotide from about 0.01 μM to about 20 μM. In a particularly preferred embodiment, the therapeutic composition is administered at a sufficient dosage to attain a blood level of antisense oligonucleotide from about 0.05 μM to about 15 μM. In a more preferred embodiment, the blood level of antisense oligonucleotide is from about 0.1 μM to about 10 μM.

For localized administration, much lower concentrations than this may be therapeutically effective. Preferably, a total dosage of antisense oligonucleotide will range from about 0.1 mg to about 200 mg oligonucleotide per kg body weight per day. In a more preferred embodiment, a total dosage of antisense oligonucleotide will range from about 1 mg to about 20 mg oligonucleotide per kg body weight per day. In a most preferred embodiment, a total dosage of antisense oligonucleotide will range from about 1 mg to about 10 mg oligonucleotide per kg body weight per day. In a particularly preferred embodiment, the therapeutically effective amount of HDAC-7 or HDAC-8 antisense oligonucleotide is about 5 mg oligonucleotide per kg body weight per day.

The method may further comprise administering to the animal a therapeutically effective amount of an HDAC-7 and/or HDAC-8 small molecule inhibitor with a pharmaceutically acceptable carrier for a therapeutically effective period of time. In some preferred embodiments, the histone deacetylase small molecule inhibitor is operably associated with the antisense oligonucleotide, as described supra.

The histone deacetylase small molecule inhibitor-containing therapeutic composition of the invention is administered systemically at a sufficient dosage to attain a blood level histone deacetylase small molecule inhibitor from about 0.01 μM to about 10 μM. In a particularly preferred embodiment, the therapeutic composition is administered at a sufficient dosage to attain a blood level of histone deacetylase small molecule inhibitor from about 0.05 μM to about 10 μM. In a more preferred embodiment, the blood level of histone deacetylase small molecule inhibitor is from about 0.1 μM to about 5 μM. For localized administration, much lower concentrations than this may be effective. Preferably, a total dosage of histone deacetylase small molecule inhibitor will range from about 0.01 mg to about 100 mg protein effector per kg body weight per day. In a more preferred embodiment, a total dosage of histone deacetylase small molecule inhibitor will range from about 0.1 mg to about 50 mg protein effector per kg body weight per day. In a most preferred embodiment, a total dosage of histone deacetylase small molecule inhibitor will range from about 0.1 mg to about 25 mg protein effector per kg body weight per day. In a particularly preferred embodiment, the therapeutically effective synergistic amount of histone deacetylase small molecule inhibitor (when administered with an antisense oligonucleotide) is about 5 mg per kg body weight per day.

When the method of the invention results in an improved inhibitory effect, the therapeutically effective concentrations of either or both of the nucleic acid level inhibitor (i.e., antisense oligonucleotide) and the protein level inhibitor (i.e.,histone deacetylase small molecule inhibitor) required to obtain a given inhibitory effect are reduced as compared to those necessary when either is used individually.

Furthermore, one of skill will appreciate that the therapeutically effective synergistic amount of either the antisense oligonucleotide or the histone deacetylase inhibitor may be lowered or increased by fine tuning and altering the amount of the other component. The invention therefore provides a method to tailor the administration/treatment to the particular exigencies specific to a given animal species or particular patient. Therapeutically effective ranges may be easily determined for example empirically by starting at relatively low amounts and by step-wise increments with concurrent evaluation of inhibition.

In a fourth aspect, the invention provides a method for inhibiting the HDAC-7 and/or HDAC-8 isoform in a cell comprising contacting the cell with a small molecule inhibitor of the first aspect of the invention. In certain preferred embodiments of the fourth aspect of the invention, cell proliferation is inhibited in the contacted cell. In preferred embodiments, the cell is a neoplastic cell which may be in an animal, including a human, and which may be in a neoplastic growth.

In a fifth aspect, the invention provides a method for inhibiting neoplastic cell growth in an animal comprising administering to an animal having at least one neoplastic cell present in its body a therapeutically effective amount of a small molecule inhibitor of the first aspect of the invention with a pharmaceutically acceptable carrier for a therapeutically effective period of time.

The histone deacetylase small molecule inhibitor-containing therapeutic composition of the invention is administered systemically at a sufficient dosage to attain a blood level histone deacetylase small molecule inhibitor from about 0.01 μM to about 10 μM. In a particularly preferred embodiment, the therapeutic composition is administered at a sufficient dosage to attain a blood level of histone deacetylase small molecule inhibitor from about 0.05 μM to about 10 μM. In a more preferred embodiment, the blood level of histone deacetylase small molecule inhibitor is from about 0.1 μM to about 5 μM. For localized administration, much lower concentrations than this may be effective. Preferably, a total dosage of histone deacetylase small molecule inhibitor ranges from about 0.01 mg to about 100 mg protein effector per kg body weight per day. In a more preferred embodiment, a total dosage of histone deacetylase small molecule inhibitor ranges from about 0.1 mg to about 50 mg protein effector per kg body weight per day. In a most preferred embodiment, a total dosage of histone deacetylase small molecule inhibitor will range from about 0.1 mg to about 25 mg protein effector per kg body weight per day.

In a sixth aspect, the invention provides a method of inhibiting the induction of cell proliferation, comprising contacting a cell with an antisense oligonucleotide that inhibits the expression of HDAC-7 or HDAC-8 or contacting a cell with a small molecule inhibitor of HDAC-7 and/or HDAC-8. In certain preferred embodiments, the cell is a neoplastic cell, and the induction of cell proliferation is tumorigenesis.

The invention further provides for histone deacetylase small molecule inhibitors that may be generated which specifically inhibit the histone deacetylase isoform(s) required for inducing cell proliferation, e.g., HDAC-7 and HDAC-8, while not inhibiting other histone deacetylase isoforms not required for inducing cell proliferation. Accordingly, in a seventh aspect, the invention provides a method for identifying a small molecule histone deacetylase inhibitor that inhibits the HDAC-7 and/or HDAC-8 isoform, which is required for the induction of cell proliferation. The method comprises contacting the HDAC-7 and/or the HDAC-8 isoform with a candidate small molecule inhibitor and measuring the enzymatic activity of the contacted histone deacetylase isoform, wherein a reduction in the enzymatic activity of the contacted histone deacetylase isoform identifies the candidate small molecule inhibitor as a small molecule histone deacetylase inhibitor that inhibits the histone deacetylase isoform, i.e., HDAC-7 and/or HDAC-8.

Measurement of the enzymatic activity of HDAC-7 or HDAC-8 may be achieved using known methodologies. For example, Yoshida et al. (J. Biol. Chem. 265:17174-17179, 1990) describe the assessment of histone deacetylase enzymatic activity by the detection of acetylated histones in trichostatin A treated cells. Taunton et al. (Science 272:408-411, 1996) similarly describes methods to measure histone deacetylase enzymatic activity using endogenous and recombinant HDAC. Both Yoshida et al. (J. Biol. Chem. 265:17174-17179, 1990) and Taunton et al. (Science 272:408-411, 1996) are hereby incorporated by reference.

Preferably, the histone deacetylase small molecule inhibitor that inhibits the HDAC-7 and/or the HDAC-8 isoform required for induction of cell proliferation is an HDAC-7 and/or HDAC-8 small molecule inhibitor that interacts with and reduces the enzymatic activity of the HDAC-7 and/or the HDAC-8 isoform.

In an eighth aspect, the invention provides a method for identifying a small molecule histone deacetylase inhibitor that inhibits the HDAC-7 and/or HDAC-8 isoform involved in the induction of cell proliferation. The method comprises contacting a cell with a candidate small molecule inhibitor and measuring the enzymatic activity of the contacted histone deacetylase isoform, wherein a reduction in the enzymatic activity of the HDAC-7 and/or HDAC-8 isoform identifies the candidate small molecule inhibitor as a small molecule histone deacetylase inhibitor that inhibits HDAC-7 and/or HDAC-8.

In a ninth aspect, the invention provides a small molecule histone deacetylase inhibitor identified by the method of the seventh or the eighth aspects of the invention. Preferably, the histone deacetylase small molecule inhibitor is substantially pure.

In a tenth aspect, the invention provides a method for inhibiting cell proliferation in a cell comprising contacting a cell with at least two reagents selected from the group consisting of an antisense oligonucleotide that inhibits expression of HDAC-7 or HDAC-8 isoform, a small molecule histone deacetylase inhibitor that inhibits expression or activity of HDAC-7 and/or HDAC-8 isoform, an anti sense oligonucleotide that inhibits expression of the HDAC-1 isoform, a small molecule histone deacetylase inhibitor that inhibits the expression or the activity of the HDAC-1 isoform, an antisense oligonucleotide that inhibits expression of a DNA methyltransferase, and a small molecule DNA methyltransferase inhibitor. In one embodiment, the inhibition of cell growth of the contacted cell is greater than the inhibition of cell growth of a cell contacted with only one of the reagents. In certain embodiments, each of the reagents selected from the group is substantially pure. In preferred embodiments, the cell is a neoplastic cell. In yet additional preferred embodiments, the reagents selected from the group are operably associated.

In an eleventh aspect, the invention provides a method of inhibiting neoplastic cell growth comprising contacting a cell with at least two reagents selected from the group consisting of an antisense oligonucleotide that inhibits expression of HDAC-7 or HDAC-8 isoform, a small molecule histone deacetylase inhibitor that inhibits the expression or the activity of HDAC-7 and/or HDAC-8 isoform, an antisense oligonucleotide that inhibits expression of the HDAC-1 isoform, a small molecule histone deacetylase inhibitor that inhibits expression or activity of the HDAC-1 isoform, an antisense oligonucleotide that inhibits expression of a DNA methyltransferase, and a small molecule DNA methyltransferase inhibitor. In one embodiment, the inhibition of cell growth of the contacted cell is greater than the inhibition of cell growth of a cell contacted with only one of the reagents that inhibit HDAC-7 or HDAC-8. In certain embodiments, each of the reagents selected from the group is substantially pure. In preferred embodiments, the cell is a neoplastic cell. In yet additional preferred embodiments, the reagents selected from the group are operably associated.

Antisense oligonucleotides that inhibit DNA methyltransferase are described in Szyf and von Hofe, U.S. Pat. No. 6,054,339. DNA methyltransferase small molecule inhibitors include, without limitation, 5-aza-2′-deoxycytidine (5-aza-dC), 5-fluoro-2′-deoxycytidine, 5-aza-cytidine (5-aza-C), or 5,6-dihydro-5-aza-cytidine.

### EXAMPLES

The following examples are intended to further illustrate certain preferred embodiments of the invention and are not limiting in nature. Those skilled in the art will recognize, or be able to ascertain, using no more than routine experimentation, numerous equivalents to the specific substances and procedures described herein. Such equivalents are considered to be within the scope of this invention, and are covered by the appended claims.

### Example 1

Expression of HDAC Isotypes in Human Clinical Samples Analyzed by Human Cancer Profiling Array

Gene expression of human HDAC1, HDAC7 and HDAC8 in human cancer and the matched normal tissues at mRNA levels was determined by using Cancer Profiling Array (Clontech, Palo Alto, Calif.). The cDNA probes of human HDAC1, HDAC7 and HDAC8 were made by PCR reactions with ³²P-labelled dCTP and primers corresponding to the 3′-end of the coding sequences of each HDAC isotypes. To PCR cDNA probe for HDAC1, the primer used corresponded to the nucleotide position #1486 to 1515 for human HDAC1 gene (accession #NM_{—}004964), with the sequence: 5′-CAT TCA GGC CAA GTC GAC CTC CTC CTT GAC-3′. To PCR HDAC7 cDNA probe, the primer used corresponded to the nucleotide position of #2858 to #2890 of human HDAC7 gene (accession #NM_{—}015401), with sequence 5′-ATG AAT TCC TGT GCA CCC GGA TCA CGG CCT CCA GAG AGC GG-3′. To PCR HDAC8 cDNA probe, the primer used corresponded to the nucleotide position of #1168-#1186 of human HDAC8 sequence (accession #AF_{—}230097), with sequence 5′-CCC TCG AGG ACC ACA TGC TTC AGA TTC-3′. Templates for PCR were purified HDAC1, HDAC7 or HDAC8 gene fragments. PCR reactions were performed using Expand™ Long Template PCR system (Roche Diagnostics Biochemical Product, Indianapolis, Ind.). Hybridization of cDNA probes for human HDAC1, HDAC7 or HDAC8 to nylon array membrane was performed as suggested by the vendor (Clontech, Palo Alto, Calif.). After hybridization and washing, array membranes were exposed to Cyclone Phosphor-Screen (Packard, Meriden, Conn.) for data analysis. Expression levels of HDAC isotypes shown in Table 1 were normalized by that of ubiquitin. As shown in Table 1, there is significant upregulation of HDAC1 expression at the RNA level in patients with uterus, ovary and lung cancers, while significant upregulation of HDAC7 or HDAC8 expression was observed in patients with colon and rectum cancers.

TABLE 1
Human HDAC Isotype mRNA Expression in Paired Normal vs.
Tumor Tissues from Patients*

| | | | | | % of patients with altered | | | | | |
|---|---|---|---|---|---|---|---|---|---|---|
| | | Expression in | | | expression in tumor tissues** | | | | | |
| # of patients | | normal tissues # | | | HDAC1 | | HDAC7 | | HDAC8 | |
| analysed | Tissue | HDAC1 | HDAC7 | HDAC8 | up | down | up | down | up | down |
| 50 | breast | ++ | ++ | ++ | 30 | 32 | 10 | 48 | 10 | 48 |
| 42 | uterus | ++ | ++ | ++ | 40 | 2 | 19 | 14 | 19 | 14 |
| 35 | colon | ++++ | ++ | ++ | 11 | 43 | 43 | 14 | 46 | 14 |
| 27 | stomach | +++ | ++ | ++ | 22 | 30 | 30 | 19 | 30 | 19 |
| 12 | ovary | ++ | ++ | ++ | 42 | 25 | 8 | 67 | 8 | 67 |
| 1 | cervix | + | + | + | 100 | 0 | 0 | 0 | 0 | 0 |
| 21 | lung | ++ | ++ | ++ | 52 | 10 | 19 | 14 | 19 | 14 |
| 20 | kidney | ++ | ++ | ++ | 10 | 70 | 0 | 75 | 0 | 75 |
| 18 | rectum | +++ | ++ | ++ | 0 | 61 | 39 | 0 | 39 | 0 |
| 2 | small intestine | +++ | ++ | ++ | 50 | 0 | 50 | 0 | 50 | 0 |
| 6 | thyroid | +++ | ++ | ++ | 17 | 33 | 17 | 0 | 17 | 0 |
| 4 | prostate | +++ | ++ | ++ | 25 | 0 | 25 | 0 | 25 | 0 |
| 1 | pancreas | ++++ | ++++ | +++ | 0 | 0 | 0 | 0 | 0 | 0 |

*Expression was analyzed by Cancer Profiling Array (Clontech); Expression of each HDAC isotype was normalized against that of ubiquitin;
#“+” means detectable; “++” means 2-4 × over “+”; “+++” means 5-9 × over “+”; “++++” means more than 10 × over “+”.
**Expression of the HDAC isotype in tumor tissues was changed at least 1.5 fold over that in the paired normal tissues.

### Example 2

Synthesis and Identification of Antisense Oligonucleotides

Antisense (AS) and mismatch (MM) oligodeoxynucleotides (oligos) were designed to be directed against the 5′- or 3′-untranslated region (UTR) of the targeted gene. Oligos listed in Table 2 were synthesized with the phosphorothioate backbone and the 4×4 nucleotides 2′-O-methyl modification on an automated synthesizer and purified by preparative reverse-phase HPLC. All oligos used were 20 base pairs in length.

To identify antisense oligodeoxynucleotide (ODN) capable of inhibiting HDAC-7 expression in human cancer cells, eighteen phosphorothioate ODNs containing sequences complementary to the 5′ or 3′ UTR of the human HDAC-7 gene (GenBank Accession No. AF239243) were initially screened in A549 cells at 100 nM. Cells were harvested after 24 hours of treatment, and HDAC-7 RNA expression was analyzed by Northern blot analysis. From the screen, we identified both AS-1 and AS-2 against human HDAC7 (see Table 2) with good antisense activities. Total RNAs were harvested and were analyzed by Northern Blot. GAPDH expression was analyzed to indicate total RNA loading in each lane.

To identify antisense oligodeoxynucleotides (ODN) capable of inhibiting HDAC-8 expression in human cancer cells, fourteen phosphorothioate ODNs containing sequences complementary to the 5′ or 3′ UTR of the human HDAC-7 gene (GenBank Accession No. AF230097) were initially screened in A549 cells at 100 nM. Cells were harvested after 24 hours of treatment, and HDAC-8 RNA expression was analyzed by Northern blot analysis. From the screen, we identified both AS-1 and AS-2 against human HDAC8 (see Table 2) with good antisense activities. Total RNAs were harvested and were analyzed by Northern Blot. GAPDH expression was analyzed to indicate total RNA loading in each lane.

To identify antisense oligodeoxynucleotide (ODN) capable of inhibiting HDAC-1 expression in human cancer cells, eleven phosphorothioate ODNs containing sequences complementary to the 5′ or 3′ UTR of the human HDAC-1 gene (GenBank Accession No. U50079) were initially screened in T24 cells at 100 nM. Cells were harvested after 24 hours of treatment, and HDAC-1 RNA expression was analyzed by Northern blot analysis. This screen identified HDAC-1 AS as an ODN with antisense activity to human HDAC-1. HDAC-1 MM oligo was created as a control; compared to the antisense oligo, it has a 6-base mismatch.

Twenty-four phosphorothioate ODNs containing sequences complementary to the 5′ or 3′ UTR of the human HDAC-2 gene (GenBank Accession No. U31814) were screened as above. HDAC-2 AS was identified as an ODN with antisense activity to human HDAC-2. HDAC-2 MM was created as a control; compared to the antisense oligo, it contains a 7-base mismatch.

Twenty-one phosphorothioate ODNs containing sequences complementary to the 5′ or 3′ UTR of the human HDAC-3 gene (GenBank Accession No. AF039703) were screened as above. HDAC-3 AS was identified as an ODN with antisense activity to human HDAC-3. HDAC-3 MM oligo was created as a control; compared to the antisense oligo, it contains a a 6-base mismatch.

Seventeen phosphorothioate ODNs containing sequences complementary to the 5′ or 3′ UTR of the human HDAC-4 gene (GenBank Accession No. AB006626) were screened as above. HDAC-4 AS was identified as an ODN with antisense activity to human HDAC-4. HDAC-4 MM oligo was created as a control; compared to the antisense oligo, it contains a 6-base mismatch.

Thirteen phosphorothloate ODNs containing sequences complementary to the 5′ or 3′ untranslated regions of the human HDAC-6 gene (GenBank Accession No. AJ011972) were screened as above. HDAC-6 AS was identified as an ODN with antisense activity to human HDAC-6. HDAC-6 MM oligo was created as a control; compared to the antisense oligo, it contains a 7-base mismatch.

As a common control of all AS ODNs, we also synthesized a 20-mer long second generation of phosphorothloate ODNs (UMM) which contain 25% of dA, 25% of dC, 25% of dG, and 25% of dT at each nucleotide position.

TABLE 2
HDAC isotype-specific antisense and mismatch oligos

| | | Accession | Nucleotide | | position |
|---|---|---|---|---|---|
| Oligo | Target | Number | Position | Sequence | within Gene |
| HDAC1 AS1 | Human HDAC1 | U50079 | 1585-1604 | 5′-GAAACGTGAGGGACTCAGCA-3′ | 3′-UTR |
| HDAC1 MM1 | Human HDAC1 | U50079 | 1585-1604 | 5′-GUUAGGTGAGGCACTGAGGA-3′ | 3′-UTR |
| HDAC1 AS2 | Human HDAC1 | U50079 | 1565-1584 | 5′-GGAAGCCAGAGCTGGAGAGG-3′ | 3′-UTR |
| HDAC2 AS | Human HDAC2 | U31814 | 1643-1662 | 5′-GCUGAGCTGTTCTGATUUGG-3′ | 3′-UTR |
| HDAC2 MM | Human HDAC2 | U31814 | 1643-1662 | 5′-CGUGAGCACTTCTCATUUCC-3′ | 3′-UTR |
| HDAC3 AS1 | Human HDAC3 | AF039703 | 1276-1295 | 5′-CGCUTTCCTTGTCATTGACA-3′ | 3′-UTR |
| HDAC3 MM1 | Human HDAC3 | AF039703 | 1276-1295 | 5′-GCCUTTCCTACTCATTGUGU-3′ | 3′-UTR |
| HDAC3 AS2 | Human HDAC3 | AF039703 | 1487-1506 | 5′-GGUACCATTGTCAGGCCUUG-3′ | 3′-UTR |
| HDAC3 MM2 | Human HDAC3 | AF039703 | 1487-1506 | 5′-CCUACCATTCACAGGCCUAC-3′ | 3′-UTR |
| HDAC4 AS1 | Human HDAC4 | AB006626 | 514-33 | 5′-GCUGCCTGCCGTGCCCACCC-3′ | 5′-UTR |
| HDAC4 MM1 | Human HDAC4 | AB006626 | 514-33 | 5′-CGUGCCTGCGCTGCCCACGG-3′ | 5′-UTR |
| HDAC4 AS2 | Human HDAC4 | AB006626 | 7710-29 | 5′-UACAGTCCATGCAACCUCCA-3′ | 3′-UTR |
| HDAC4 MM2 | Human HDAC4 | AB006626 | 7710-29 | 5′-AUCAGTCCAACCAACCUCGU-3′ | 3′-UTR |
| HDAC5 AS1 | Human HDAC5 | BE794912 | 1-20 | 5′-GCAGCGGCGGCAGCACCUCC-3′ | 5′-UTR |
| HDAC5 AS2 | Human HDAC5 | AF039691 | 2663-2682 | 5′-CTTCGGTCTCACCTGCTTGG-3′ | 3′-UTR |
| HDAC5 AS3 | Human HDAC5 | BE794912 | 259-278 | 5′-CGUUGGGAGAGTTCATGCCG-3′ | 5′-UTR |
| HDAC6 AS | Human HDAC6 | AJ011972 | 3791-3810 | 5′-CAGGCTGGAATGAGCTACAG-3′ | 3′-UTR |
| HDAC6 MM | Human HDAC6 | AJ011972 | 3791-3810 | 5′-GACGCTGCAATCAGGTAGAC-3′ | 3′-UTR |
| HDAC7 AS1 | Human HDAC7 | AF239243 | 65-84 | 5′-CAGGCTCACTTGACAAUGGC-3′ | 5′-UTR |
| HDAC7 MM1 | Human HDAC7 | AF239243 | 65-84 | 5′-GUGGCACACAAGACAAUCCC-3′ | 5′-UTR |
| HDAC7 AS2 | Human HDAC7 | AF239243 | 2896-2915 | 5′-CUUCAGCCAGGATGCCCACA-3′ | 3′-UTR |
| HDAC8 AS1 | Human HDAC8 | AF230097 | 51-70 | 5′-CUCCGGCTCCTCCATCUUCC-3′ | 5′-UTR |
| HDAC8 MM1 | Human HDAC8 | AF230097 | 51-70 | 5′-GACCGGCTGCACCATCTTGG-3′ | 5′-UTR |
| HDAC8 AS2 | Human HDAC8 | AF230097 | 1328-1347 | 5′-AGCCAGCTGCCACTTGAUGC-3′ | 3′-UTR |
| HDAC8 MM2 | Human HDAC8 | AF230097 | 1328-1347 | 5′-UCCCAGCTGGCTCTTGAAGG-5′ | 3′-UTR |
| UMM* | | | | 5′-NNNNNNNNNNNNNNNNNNNN-3′ | |

*UMM is a second generation phosphorothioate oligo with 4 X 4 2′-O-methyl modification.
Each nucleotide position (N) contains 25% of dA, 25% of dT, 25% of dG, 25% of dC

### Example 3

HDAC AS ODNs Specifically Inhibit Expression at the mRNA Level

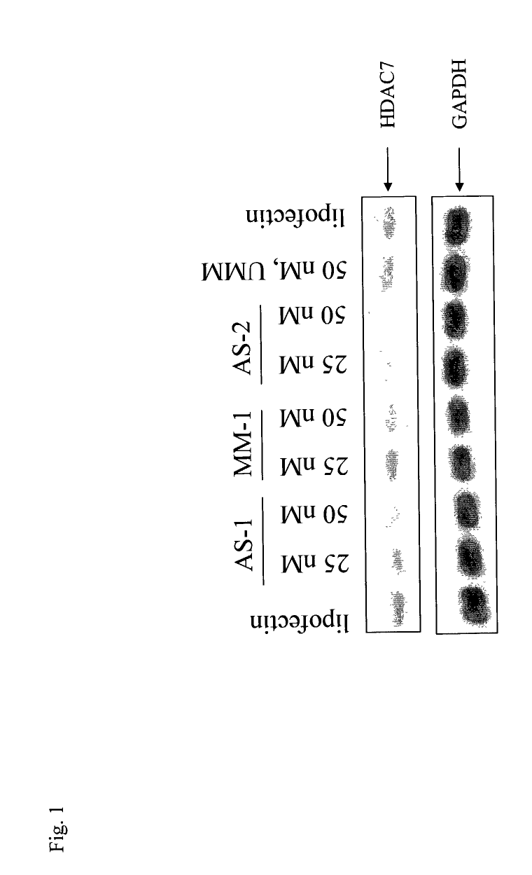

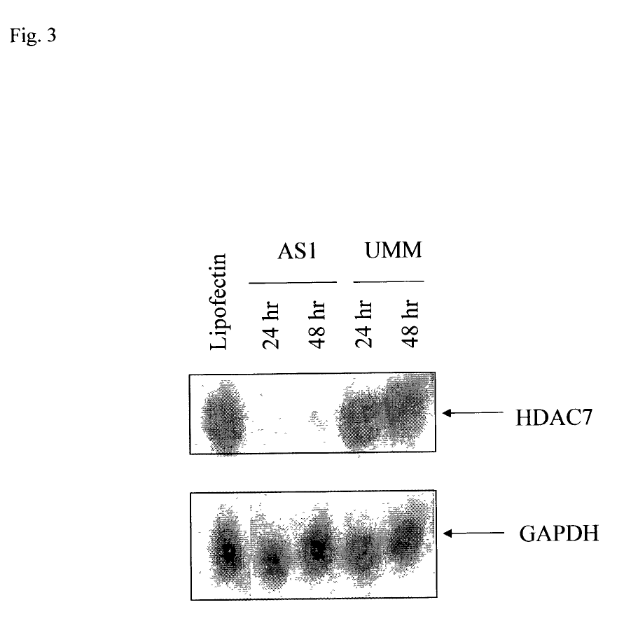

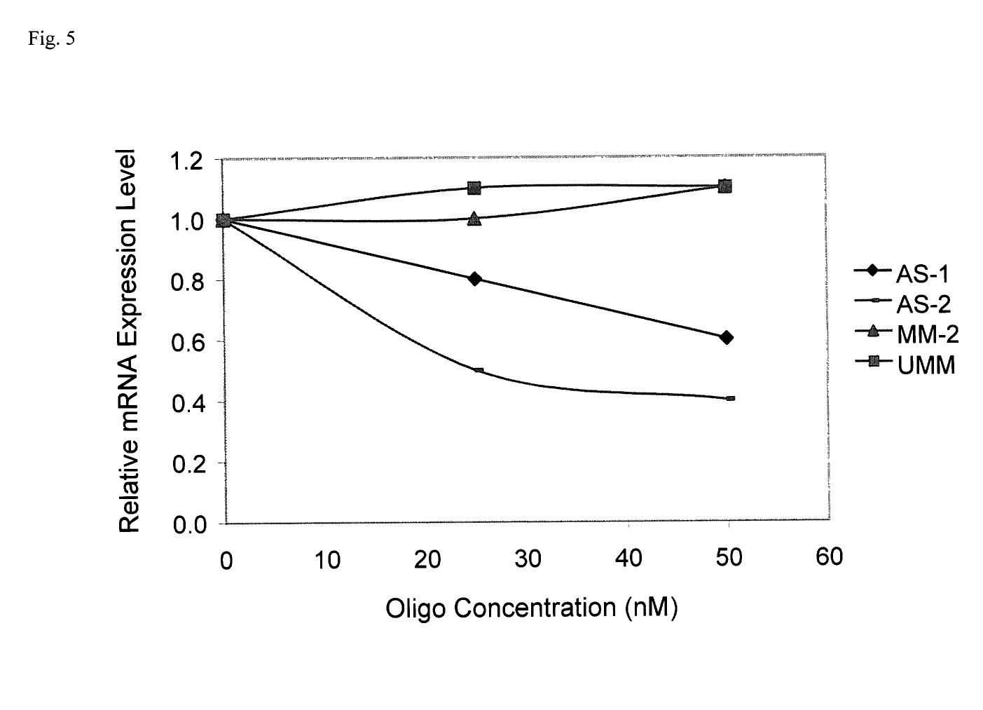

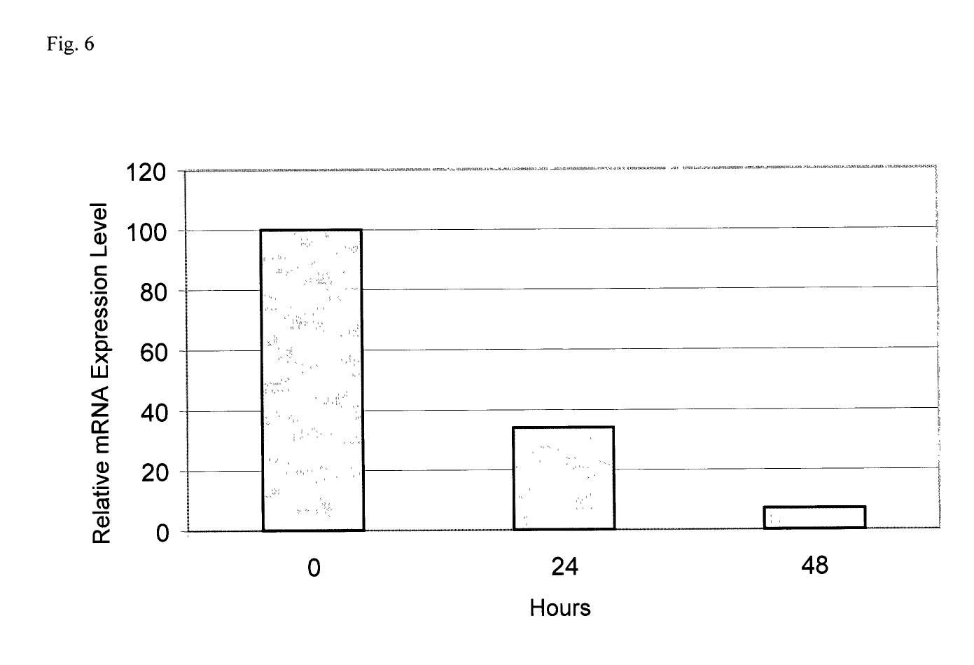

In order to determine the dose response of HDAC7 antisense inhibitors to reduce HDAC7 message at the mRNA level, Human A549 cells were treated with 25 or 50 nM of antisense (AS1 and AS2) oligos directed against human HDAC-7 or the corresponding mismatch of AS1 (MM1) oligo or an universal mismatch (UMM) for 24 hours. Shown in FIG. 1, both AS1 or AS2 can inhibit human HDAC7 expression at the mRNA level. The time dependence of HDAC7 antisense inhibitors on blocking HDAC7 gene expression at the mRNA level was analyzed by treating A549 cells with 50 nM AS1 or MM1 oligos. Shown in FIG. 3, AS1 oligo can significantly block gene expression of human HDAC7 at the mRNA level by 24 hours. Similarly, A549 cells were treated with 25 nM or 50 nM of AS1 or AS2 oligos directed against human HDAC-8 or its MM oligo for AS2 (MM2) for 24 hours. The dose response of these oligos on inhibiting HDAC8 expression at mRNA level was shown in FIG. 5. AS-2 oligo against human HDAC8 at 50 nM was also used to treat A549 cells for 24 or 48 hours. Shown in FIG. 6, AS-2 oligo significantly block HDAC8 expression at the mRNA level by 24 hours.

For all ex vivo oligo treatment, human A549 human bladder carcinoma cells were seeded in 10 cm tissue culture dishes one day prior to oligonucleotide treatment. The cell lines were obtained from the American Type Culture Collection (ATCC) (Manassas, Va.) and were grown under the recommended culture conditions. Before the addition of the oligonucleotides, cells were washed with PBS (phosphate buffered saline). Next, lipofectin transfection reagent (GIBCO BRL Mississauga, Ontario, Calif.), at a concentration of 6.25 μg/ ml, was added to serum free OPTIMEM medium (GIBCO BRL, Rockville, Md.), which was then added to the cells. The oligonucleotides were added directly to the cells (i.e., one oligonucleotide per plate of cells). Mismatched oligonucleotides were used as controls. The same concentration of oligonucleotide (e.g., 50 nM) was used per plate of cells for each oligonucleotide tested.

Cells were harvested, and total RNAs were analyzed by Northern blot analysis. Briefly, total RNA was extracted using RNeasy miniprep columns (QIAGEN Canada, Mississauga, Ontario). Ten to twenty pg of total RNA was run on a formaldehyde-containing 1% agarose gel with 0.5 M sodium phosphate (pH 7.0) as the buffer system. RNAs were then transferred to nitrocellulose membranes and hybridized with the indicated radiolabeled DNA probes. Autoradiography was performed using conventional procedures.

Our results indicate that HDAC AS ODNs can specifically inhibit targeted HDAC expression at the mRNA level.

### Example 4

HDAC OSDNs Inhibit HDAC Protein Expression

In order to determine whether treatment with HDAC ODNs would inhibit HDAC protein expression, human A549 cancer cells were treated with 25 or 50 nM of paired antisense or its mismatch oligos directed against human HDAC-7 for 48 hours. ODN treatment conditions were as previously described. To analyze the time course of AS oligos on inhibition of HDAC7 protein expression, A549 cells were treated with oligos (AS1, AS2 or UMM, each 50 nM) for either 24 hours or 48 hours.

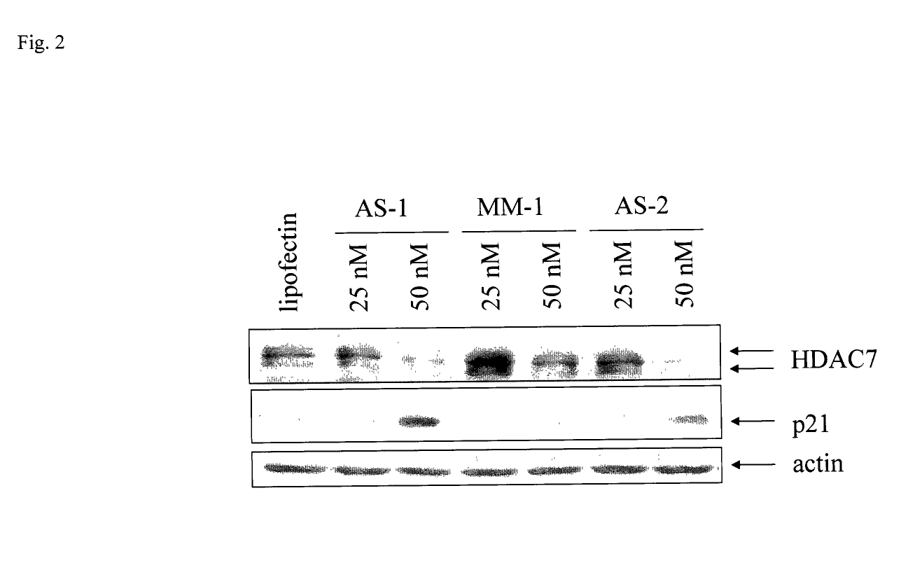

Cells were lysed in buffer containing 1% Triton X-100, 0.5% sodium deoxycholate, 5 mM EDTA, 25 mM Tris-HCl, pH 7.5, plus protease inhibitors. Total protein was quantified by the protein assay reagent from Bio-Rad (Hercules, Calif.). 100 ug of total protein was analyzed by SDS-PAGE. Next, total protein was transferred onto a PVDF membrane and probed with HDAC-7-specific primary antibodies. As shown in FIG. 2, the treatment of cells with HDAC-7 ODNs for 48 hours specifically inhibits the expression of HDAC-7 isotype protein.

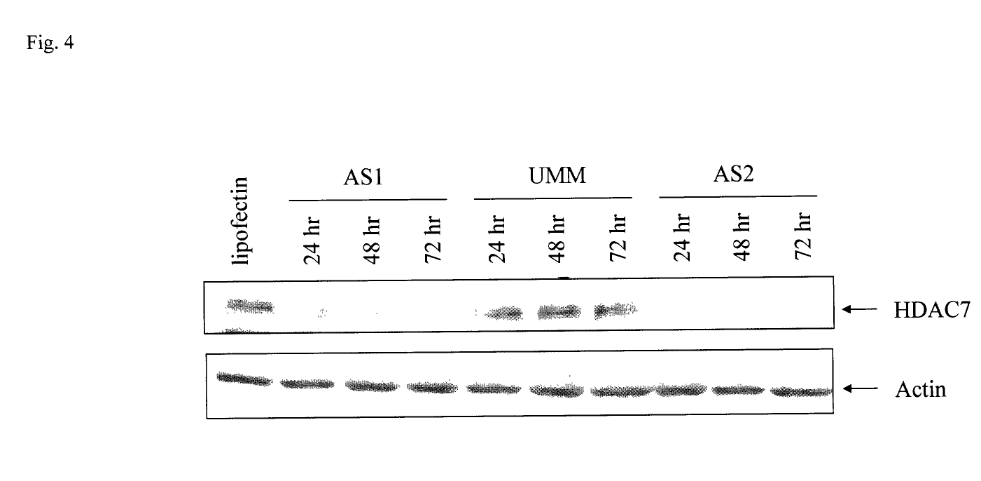

Results from FIG. 2 and FIG. 4 clearly demonstrate that HDAC7 AS oligos can inhibit expression of human HDAC7 at the protein level.

### Example 5

Effect of HDAC Isotype Specific OSDNs on Cell Growth and Apoptosis

In order to determine the effect of HDAC ODNs on cell growth inhibition and cell death through apoptosis, A549, T24, DuI45, HCT116 cells (ATCC, Manassas, Va.), or HMEC cells (BioWhittaker, Walkersville, Md.) were treated with HDAC ODNs as previously described.

For the apoptosis study, cells were analyzed using the Cell Death Detection ELISA^{Plus} kit (Roche Diagnostic GmBH, Mannheim, Germany) according to the manufacturer's directions. Typically, 10,000 cells were plated in 96-well tissue culture dishes for 2 hours before harvest and lysis. Each sample was analyzed in duplicate. ELISA reading was done using a MR700 plate reader (DYNEX Technology, Ashford, Middlesex, England) at 410 nm. The reference was set at 490 nm.

For the cell growth inhibition analysis, human cancer or normal cells were treated with 50 nM of paired AS or MM oligos directed against human HDAC-1, HDAC-7 or HDAC-8for up to 96 hours. Cell numbers were counted every day by trypan blue exclusion. Percentage of inhibition was calculated as (100 - AS cell numbers/control cell numbers)%.

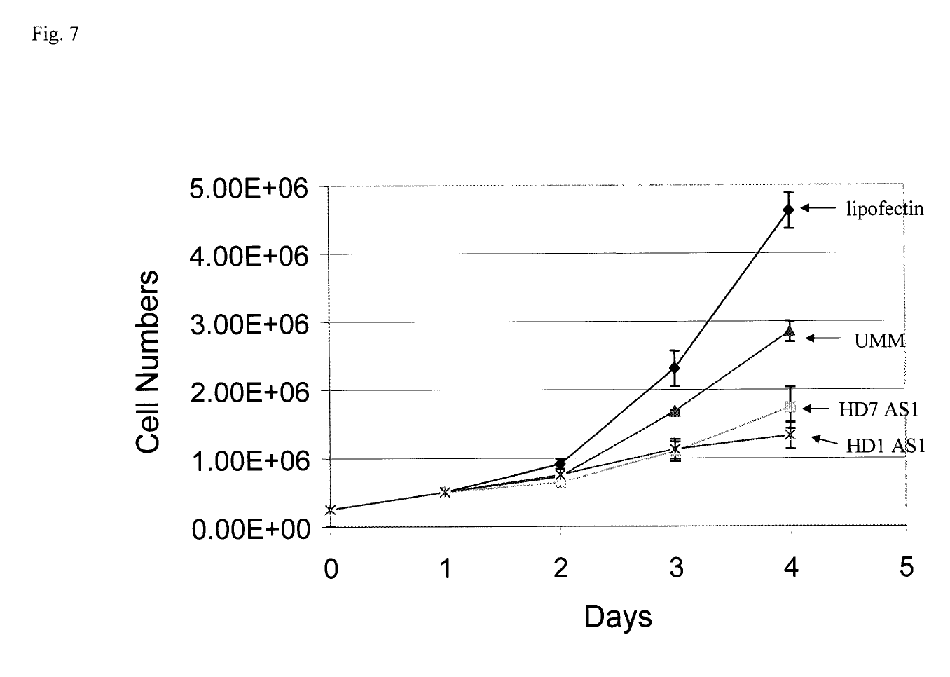

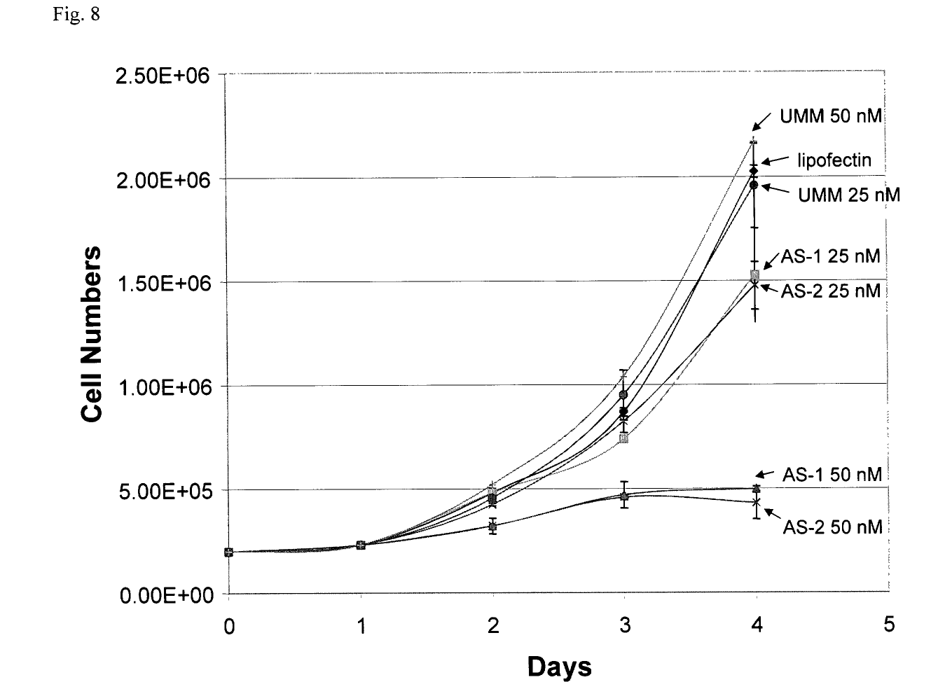

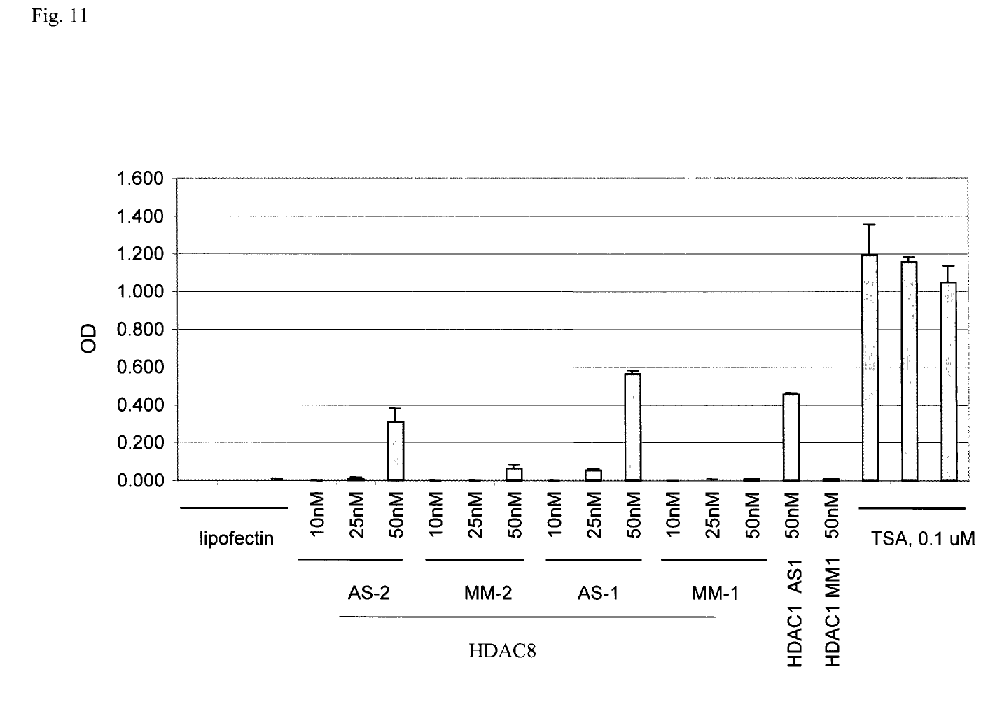

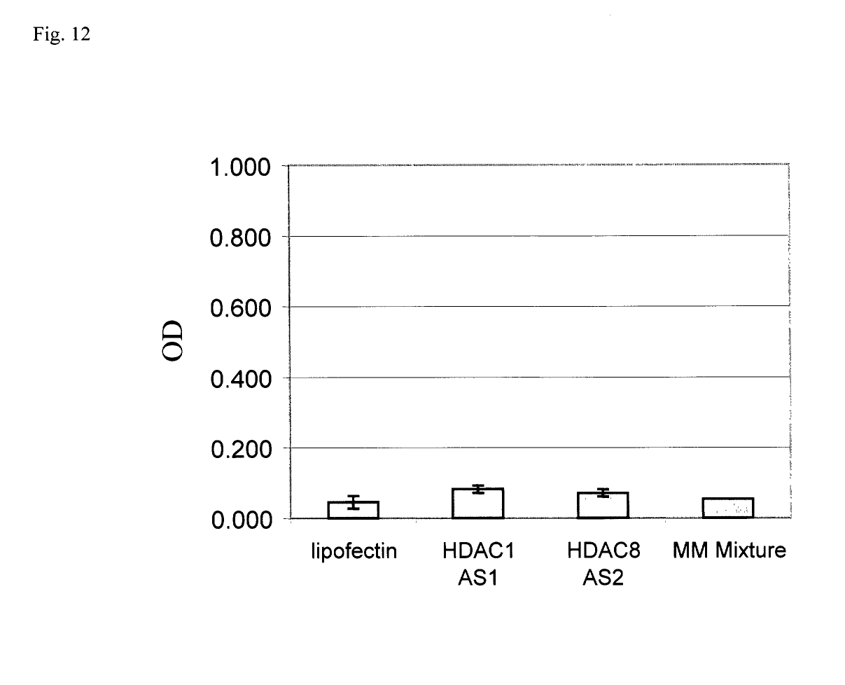

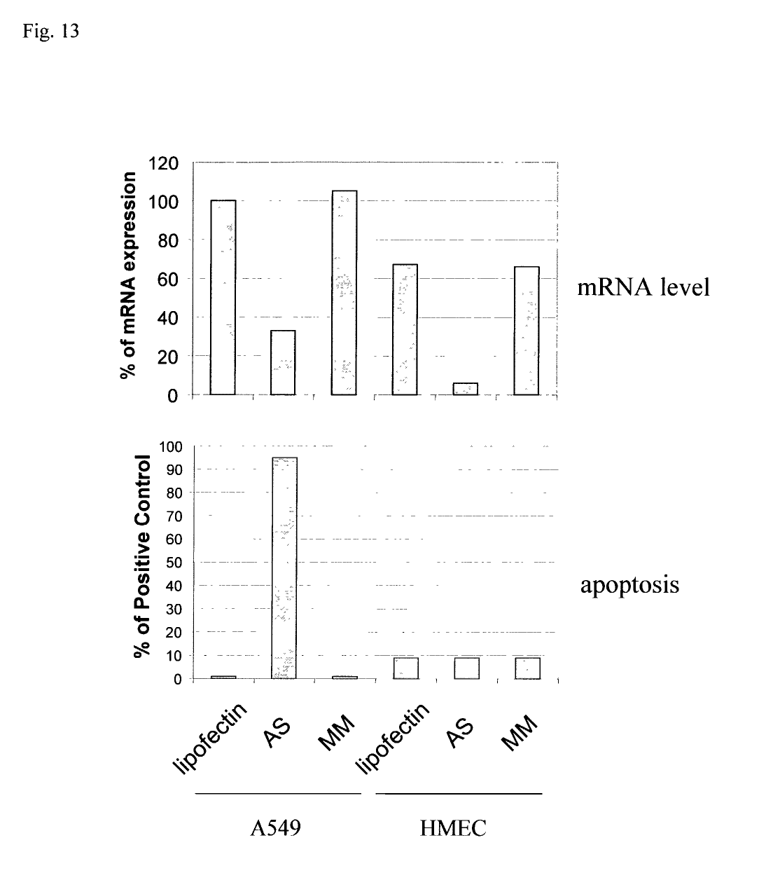

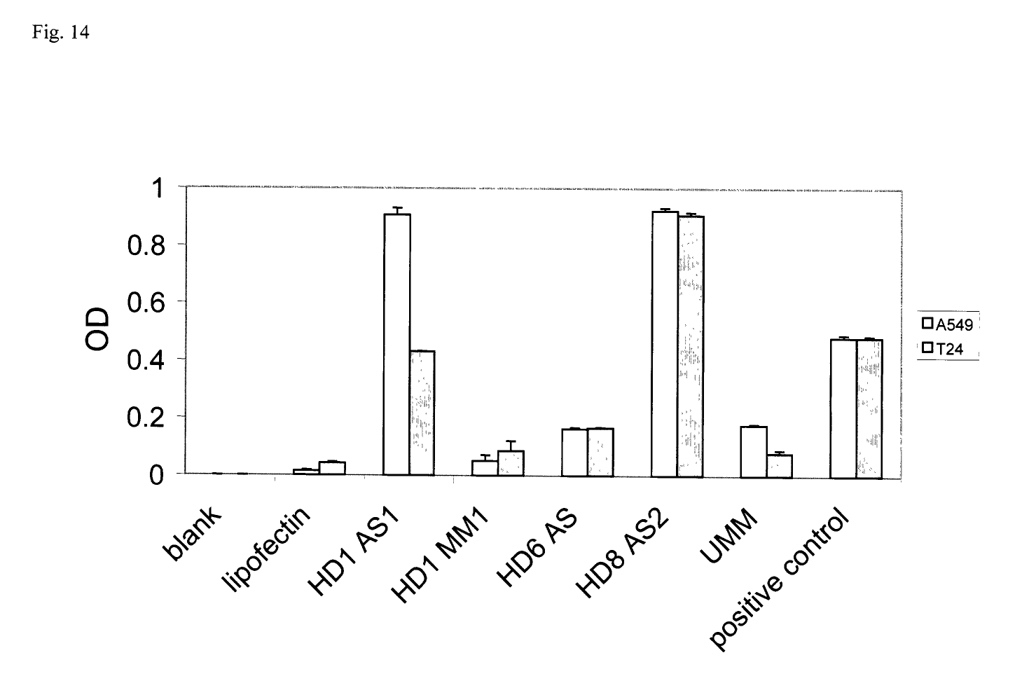

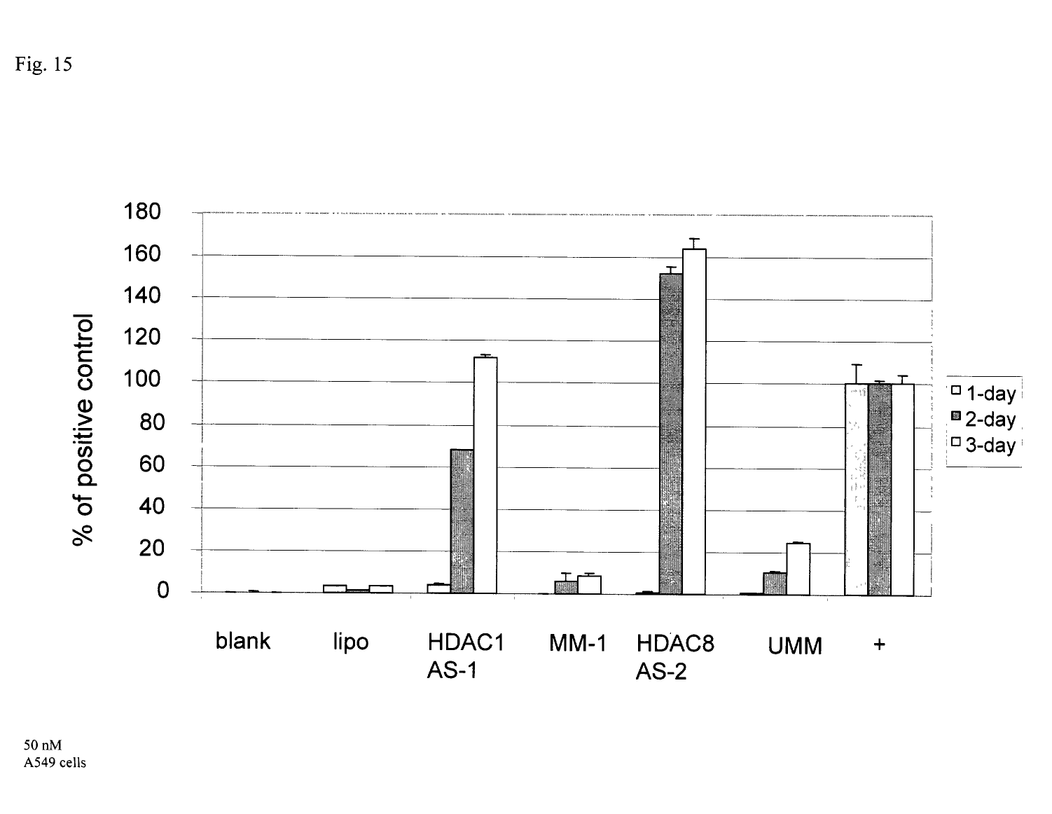

Results of the study are shown in FIGS. 7, 8, 11-16, and in Table 3. Treatment of human cancer cells by HDAC1, HDAC-7 AS, and HDAC 8 AS induces growth arrest of various human cancer cells. Treatment of human cancer cells by HDAC1 or HDAC-8 AS induces growth arrest of various human cancer cells but not normal cells. The corresponding mismatches have no effect. Since T24 cells are p53 null and A549 cells are p53 wild type, this induction of apoptosis is independent of p53 activity.

TABLE 3
Phenotypic analysis of human cancer cells treated with HDAC
Isotype-Specific Antisense Inhibitors

| | Growth | | | | | | | | | | | | inhibition of agar | | |
|---|---|---|---|---|---|---|---|---|---|---|---|---|---|---|---|
| | Inhibition* | | | | | | | Apoptosis** | | | | | colony formation*** | | |
| | | | | HCT | MCF- | | MDAmb | | | HCT | | MDAmb | | HCT | MDAmb |
| AS | A549 | T24 | Du145 | 116 | 7# | H446# | 231 | A549 | T24 | 116 | MCF-7 | 231 | A549 | 116 | 231 |
| HD1 AS1 | + | + | + | − | + | + | + | + | + | + | + | + | + | − | + |
| HD2 AS | − | − | + | | | | − | − | − | | | − | − | | − |
| HD3 AS1 | − | − | + | | | | − | − | − | | | − | − | | − |
| HD4 AS1 | + | | + | + | | | + | + | + | | | + | − | + | + |
| HD5 AS1 | − | | + | − | | | | − | | | | | − | − | |
| HD6 AS | − | − | − | | | | − | − | − | | | − | − | | − |
| HD7 AS1 | + | | + | − | | | | − | | | | | | − | |
| HD8 AS2 | + | − | + | − | | | | + | + | | | | | + | |

*“+” means >35% inhibition of cell growth by antisense oligos (50 nM, 48 hour) compared to that of cells treated with lipofectin;
**“+” means the induction of apoptosis at least 2 fold over mismatch control (50 nM, 48 hours);
***“+” means >35% inhibition of agar colony formation of cells treated with 50 nM oligos for 48 hours compared to that of cells treated with lipofectin
#75 nM treatment for 2 days

### Example 6

Synergy of Isotype-Specific Antisense Inhibitors Directed Against Human HDAC7 or HDAC8 with Antisense Inhibitor Directed Against Human HDAC1 on Induction of Apopotosis of Human Cancer Cells

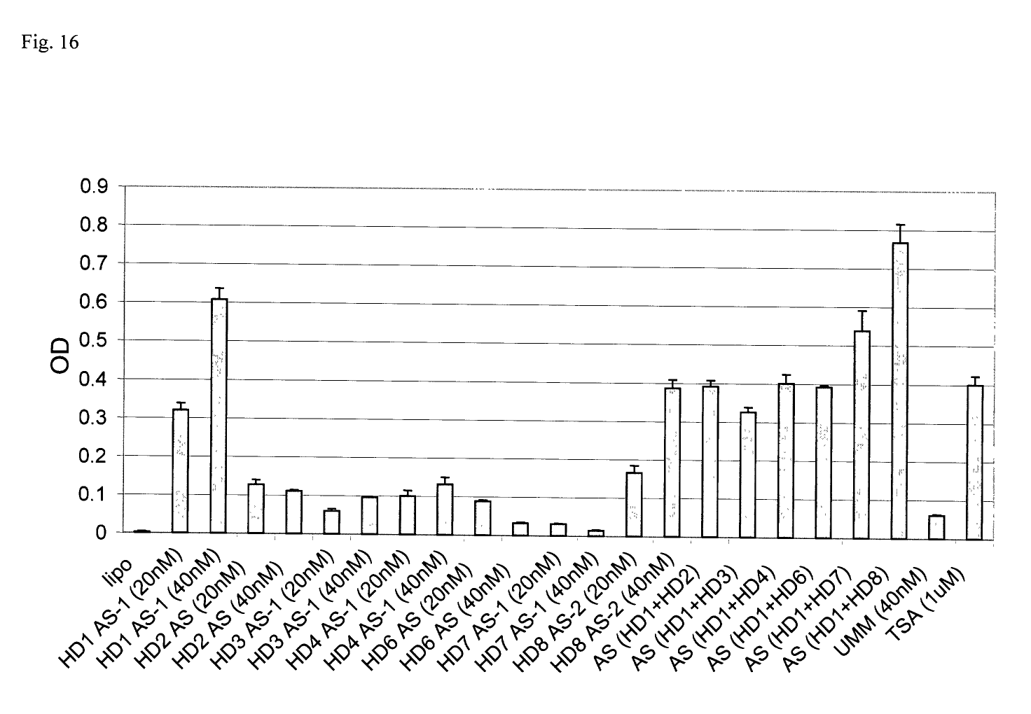

Human A549 cells were treated with each isotype-specific AS inhibitors against human HDAC 1-8 at 40 nM alone, or with 20 nM of HDAC AS oligos in addition to 20 nM of UMM control oligo, or with 40 nM of UMM control oligo. Similarly, A549 cells were treated with 20 nM of HDAC1 AS in combination with 20 nM of each of AS inhibitors against human HDAC2 to HDAC8. After 48 hour treatment, A549 cells were harvested and analyzed for apoptosis by ELISA as described previously. Apoptosis of A549 cells by AS inhibition was compared to that of cells treated with 1 uM TSA for 16 hours. Shown in FIG. 16, HDAC7 and HDAC8 AS inhibitors can synergize with HDAC1 AS inhibitor to induce significant apoptosis of human cancer A549 cells, while AS inhibitors against other HDAC isotypes did not synergize with HDAC1 AS. The control oligo UMM had no effect on induction of apoptosis. Specific inhibition of HDAC7 with HDAC1 or inhibition of HDAC8 with HDAC1 by their AS inhibitors resulted in even more dramatic induction of apoptosis in A549 cells than that by TSA treatment.

### Example 7

Effect of HDAC Isotype-Specific Antisense Inhibitors on Cell Cycle Blocks of Human Cancer Cells.

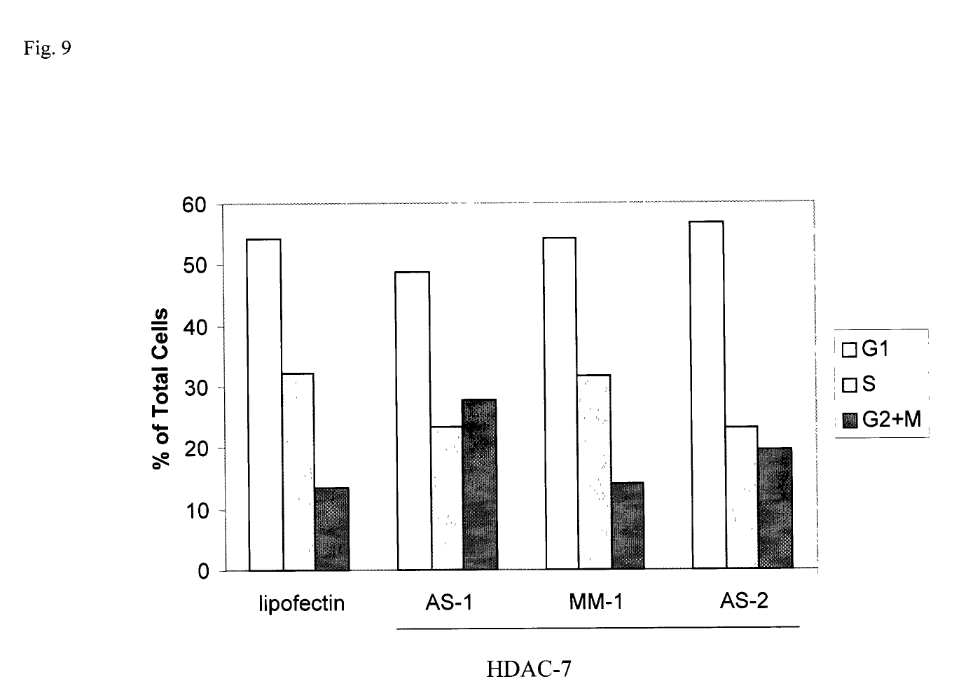

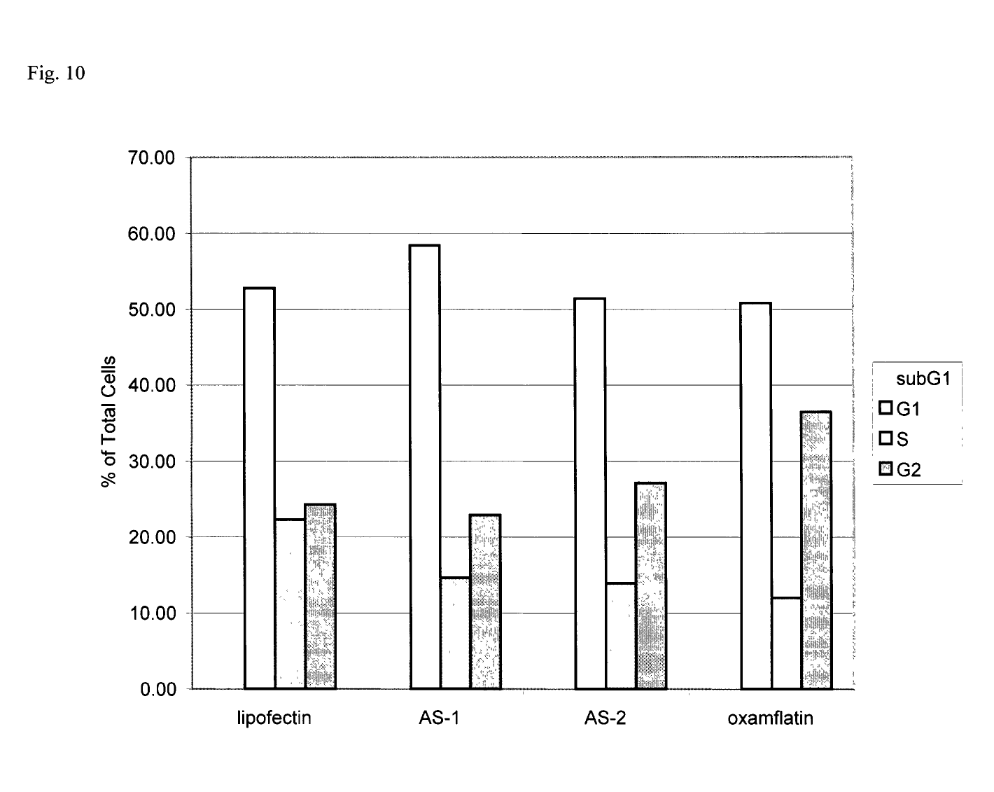

Human cancer cells (typically A549 cells) were treated with HDAC isotype-specific antisense ODNs or their mismatch control ODNs for 48 hours. Cells were harvested and fixed by 70% ethanol at −20° C. Nucleic acids from fixed cells were stained with propidium iodide (50 μg/ml). Cell cycle profiles of treated cancer cells were measured by using a fluorescence-activated cell sorter (FACScan, from Becton Dickson Immunocytometry Systems, San Jose, Calif.). Shown in FIGS. 9 and 10, antisense inhibitors of human HDAC7 or HDAC8 clearly induced cell cycle blocks of human cancer A549 cells at G2/M phase.

### Example 8

Effect of HDAC Isotype-Specific Small Molecule Inhibitors on Growth Inhibition of Various Human Cancer Cell in vitro

TABLE 4
Enzyme Inhibitory Activity and Antitumor Activity of MG HDAC
Inhibitors In vitro and an vivo (Results shown in uM)

| | | IC50 | | | | | | | |
|---|---|---|---|---|---|---|---|---|---|
| | | HD | | HD | HD | HD | HCT | Du | A |
| Cpd # | structure | 1 | HD4 | 6 | 7 | 8 | 116 | 145 | 549 |
| 4 | 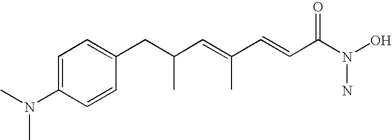 | 2 | >10 | | | 0.4 | 6 | | |
| 5 | 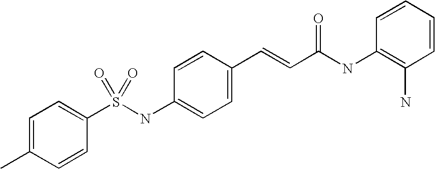 | 3 | 28 | >50 | 20 | >50 | 4 | 2 | 8 |
| 13 | 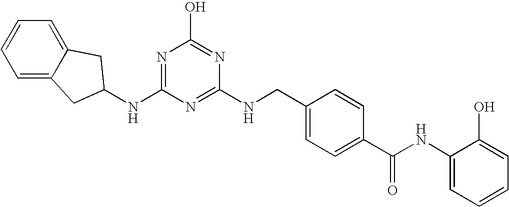 | 0.4 | >50 | >50 | | 35 | 0.5 | 0.9 | 3 |
| 16 | 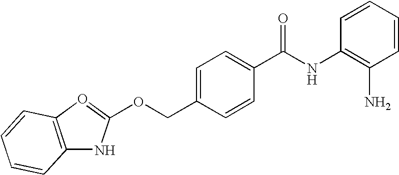 | 0.3 | 29 | >50 | 38 | | 0.2 | 0.7 | 2 |
| 17 | 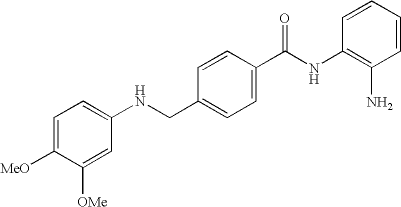 | 2 | >50 | >50 | 45 | >50 | 0.4 | 2 | 3 |
| 21 | 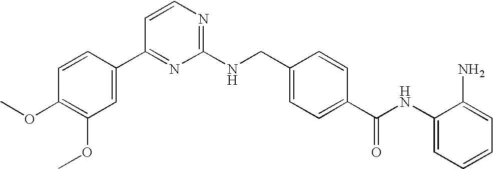 | 4 | >50 | >50 | 37 | >50 | 0.1 | 0.5 | 0.3 |

| | | | | | | % of | | | | | |
|---|---|---|---|---|---|---|---|---|---|---|---|
| | | | | | | Inhibition | | | | | |
| | | | | | | of Tumor | | | | | |
| | | | | | | Growth in | | | | | |
| | MCF | MTTIC50 | H | SW | T | Vivo | SW | A | PANC- | ES | DU |
| Cpd # | 7 | MDAmb231 | 446 | 48 | 24 | HCT116 | 48 | 549 | 1 | 2 | 145 |
| 4 | | | | | | | | | | | |
| 5 | 6 | 2 | 1 | 10 | 8 | 48(20,I) | | | | | |
| | | | | | | 55(40,I) | | | | | |
| 13 | 3 | 2 | 1 | 3 | 2 | | | | | | |
| 16 | 3 | 1 | 1 | 2 | 1 | 61(20,I) | | | | | |
| 17 | 3 | 2 | 0.9 | 2 | 2 | 77(20,I) | 68(6,0,O) | 67(5,0,O) | 78(60,O) | | >50(60,I) |
| 21 | 0.7 | 0.5 | 0.3 | 0.8 | | | | >50(30,I) | >50(3,0,I) | | |

Effects of HDAC isotype-specific small molecule inhibitors on growth inhibition of various human cancer cells (from ATCC) in vitro were determined by MTT assays. Briefly, cells seeded in 96-well plates were incubated for 72 hours at 37° C. in a 5% CO₂ incubator. MTT (Sigma) was added at a final concentration of 0.5 mg/ml and incubated with the cells for 4 hours before an equal volume of solubilization buffer (50% N,N-dimethylformamide, 20% SDS, pH 4.7) was added onto cultured cells. After overnight incubation, solubilized dye was quantified by colorimetric reading at 570 nM using a reference at 630 nM. OD values were converted to cell numbers according to a standard growth curve of the relevant cell line. The concentration which reduces cell numbers to 50% of those of DMSO-treated cells is determined as MTT IC₅₀. In Table 4, IC50s of several HDAC7 or HDAC8 inhibitors in MTT assays in various human cancer cell lines were listed. They include colon cancer cells HCT116 and SW48, lung cancer cells A549 and H446, breast cancer cells MCF-7 and MDAmb231, a prostate cancer cell line Du145 and a bladder cancer cell line T24. As shown in Table 4, all molecules can inhibit growth of human cancer cells in vitro.

### Example 9

Inhibition by Small Molecules of Tumor Growth in a Mouse Model

Female BALB/c nude mice are obtained from Charles River Laboratories (Charles River, N.Y.) and used at age 8-10 weeks. Human tumor cells (2×10⁶, colon carcinoma cells HCT116 or SW48, lung carcinoma cells A549, pancreatic carcinoma Panc-1, ovarian carcinoma cells ES2, or prostate carcinoma cells Du145) are injected subcutaneously in the animal's flank and allowed to form solid tumors. Tumor fragments are serially passaged a minimum of three times, then approximately 30 mg tumor fragments are implanted subcutaneously through a small surgical incision under general anaesthesia. Small molecule inhibitor administration by intraperotineal or oral administration was initiated when the tumors reached a volume of 100 mm³. For intraperotineal administration, small molecule inhibitors of HDAC-7, HDAC-8 or both, or in combination with small molecule inhibitors of HDAC-1 (20-60 mg/kg body weight/day) are dissolved in 100% DMSO and administered daily by injection. For oral administration, small molecule inhibitors of HDAC (60 mg/kg body weight) are dissolved in saline acidified with 0.2 N HCl. Tumor volumes are monitored twice weekly up to 20 days. Each experimental group contains at least 6-8 animals. Percentage inhibition is calculated using volume of tumor from vehicle-treated mice as controls. Shown in Table 4, inhibition of HDAC7 or HDAC8 in combination with HDAC1 leads to inhibition of growth of various human tumors in vivo.

Equivalents

Those skilled in the art will recognize, or be able to ascertain, using no more than routine experimentation, many equivalents to the specific embodiments of the invention described herein. Such equivalents are intended to be encompasssed by the following claims.

What is claimed is:

1. A method of inhibiting HDAC-7 activity in a cell, comprising contacting the cell with an antisense oligonucleotide complementary to a region of RNA that encodes a portion of HDAC-7, whereby HDAC-7 activity is inhibited.

2. The method according to claim 1, wherein the cell is contacted with an HDAC-7 antisense oligonucleotide that is a chimeric oligonucleotide.

3. The method according to claim 1, wherein the cell is contacted with an HDAC-7 antisense oligonucleotide that is a hybrid oligonucleotide.

4. The method according to claim 1, wherein the antisense oligonucleotide has a nucleotide sequence of from about 13 to about 35 nucleotides which is selected from the nucleotide sequence of SEQ ID NO: 1.

5. The method according to claim 1, wherein the antisense oligonucleotide has a nucleotide sequence of from about 15 to about 26 nucleotides which is selected from the nucleotide sequence of SEQ ID NO: 1.

6. The method according to claim 1, wherein the cell is contacted with an HDAC-8 antisense oligonucleotide that is SEQ ID NO: 3.

7. The method according to claim 1, whereby inhibition of HDAC-7 activity in the contacted cell further leads to an inhibition of cell proliferation in the contacted cell.

8. The method according to claim 1, wherein inhibition of HDAC-7 activity in the contacted cell further leads to growth retardation of the contacted cell.

9. The method according to claim 1, wherein inhibition of HDAC-7 activity in the contacted cell further leads to growth arrest of the contacted cell.

10. The method according to claim 8, wherein inhibition of HDAC-7 activity in the contacted cell further leads to necrotic cell death of the contacted cell.

11. The method according to claim 8, wherein inhibition of HDAC-8 activity in the contacted cell further leads to necrotic cell death of the contacted cell.

12. A method of inhibiting HDAC-7 or HDAC-8 activity in a cell, comprising contacting the cell with a small molecule inhibitor of HDAC-7 selected from the group consisting of:
N-Hydroxy-4,6-dimethyl-7-[(4-N,N-dimethylaminophenyl)]-2,4-heptadienamide,
N-(2-Aminophenyl)-3-[4-(4-methylbenzenesulfonylamino)-phenyl]-acrylamide,
4-{[4-Amino-6-(2-indanyl-amino)-[1,3,5]-triazin-2-yl-amino]-methyl}-N-(2-amino-phenyl)-benzamide,
N-(2-Amino-phenyl)-4-(1H-benzimidazol-2-ylsulfanylmethyl)-benzamide,
N-(2-Aminophenyl)-4-[(3,4-dimethoxyphenylamino)-methyl]-benzamide, and
N-(2-Amino-phenyl)-4-{[4-(3,4-dimethoxy-phenyl)-pyrimidin-2-ylamino]-methyl}-benzamide.

13. The method according to claim 12, whereby inhibition of HDAC-7 or HDAC-8 activity in the contacted cell further leads to an inhibition of cell proliferation in the contacted cell.

14. The method according to claim 12, wherein inhibition of HDAC-7 or HDAC-8 activity in the contacted cell further leads to growth retardation of the contacted cell.

15. The method according to claim 12, wherein inhibition of HDAC-7 or HDAC-8 activity in the contacted cell further leads to growth arrest of the contacted cell.

16. The method according to claim 12, wherein inhibition of HDAC-7 or HDAC-8 activity in the contacted cell further leads to programmed cell death of the contacted cell.

17. The method according to claim 13, wherein inhibition of HDAC-7 or HDAC-8 activity in the contacted cell further leads to necrotic cell death of the contacted cell.

18. A method for inhibiting neoplastic cell proliferation in an animal, comprising administering to an animal having at least one neoplastic cell present in its body a therapeutically effective amount of an antisense oligonucleotide complementary to a region of RNA that encodes a portion of HDAC-7 or HDAC-8, whereby neoplastic cell proliferation is inhibited.

19. The method according to claim 18, wherein the animal is administered a chimeric HDAC-7 or HDAC-8 antisense oligonucleotide.

20. The method according to claim 18, wherein the animal is administered a hybrid HDAC-7 or HDAC-8 antisense oligonucleotide.

21. The method according to claim 18, wherein the antisense oligonucleotide has a nucleotide sequence of from about 13 to about 35 nucleotides which is selected from the nucleotide sequence of SEQ ID NO: 1.

22. The method according to claim 18, wherein the antisense oligonucleotide has a nucleotide sequence of from about 15 to about 26 nucleotides which is selected from the nucleotide sequence of SEQ ID NO: 1.

23. The method according to claim 18, wherein the antisense oligonucleotide has a nucleotide sequence of from about 20 to about 26 nucleotides which is selected from the nucleotide sequence of SEQ ID NO: 2.

24. The method according to claim 18, wherein the cell is contacted with an HDAC-8 antisense oligonucleotide that is SEQ ID NO: 3.

25. The method according to claim 18, whereby inhibition of HDAC-8 activity in the contacted cell further leads to an inhibition of cell proliferation in the contacted cell.

26. The method according to claim 18, wherein inhibition of HDAC-8 activity in the contacted cell further leads to growth retardation of the contacted cell.

27. The method according to claim 18, wherein inhibition of HDAC-8 activity in the contacted cell further leads to growth arrest of the contacted cell.

28. The method according to claim 18, wherein inhibition of HDAC-8 activity in the contacted cell further leads to programmed cell death of the contacted cell.

29. The method according to claim 25, wherein inhibition of HDAC-8 activity in the contacted cell further leads to necrotic cell death of the contacted cell.

30. A method for inhibiting neoplastic cell proliferation in an animal, comprising administering to an animal having at least one neoplastic cell present in its body a therapeutically effective amount of a small molecule inhibitor of HDAC-7 or HDAC-8

31. The method according to claim 30, whereby inhibition of HDAC-7 or HDAC-8 activity in the contacted cell further leads to an inhibition of cell proliferation in the contacted cell.

32. The method according to claim 30, wherein inhibition of HDAC-7 or HDAC-8 activity in the contacted cell further leads to growth retardation of the contacted cell.

33. The method according to claim 30, wherein inhibition of HDAC-7 or HDAC-8 activity in the contacted cell further leads to growth arrest of the contacted cell.

34. The method according to claim 30, wherein inhibition of HDAC-7 or HDAC-8 activity in the contacted cell further leads to programmed cell death of the contacted cell.

35. The method according to claim 31, wherein inhibition of HDAC-7 or HDAC-8 activity in the contacted cell further leads to necrotic cell death of the contacted cell.

36. The method according to claim 18 or 30, wherein the animal is a human.

37. The method according to claim 18 or 30, further comprising administering to the animal a therapeutically effective amount of an antisense oligonucleotide complementary to a region of nucleic acid that encodes a portion of HDAC-1.

38. The method according to claim 37, wherein the animal is administered a chimeric HDAC-1 antisense oligonucleotide.

39. The method according to claim 37, wherein the animal is administered a hybrid HDAC-1 antisense oligonucleotide.

40. The method according to claim 37, wherein the animal is administered an HDAC-1 antisense oligonucleotide having a nucleotide sequence of from about 13 to about 35 nucleotides which is selected from the nucleotide sequence of SEQ ID NO: 1.

41. The method according to claim 37, wherein the animal is administered an HDAC-1 antisense oligonucleotide having a nucleotide sequence of from about 15 to about 26 nucleotides which is selected from the nucleotide sequence of SEQ ID NO: 1.

42. The method according to claim 37, wherein the animal is administered an HDAC-1 antisense oligonucleotide having a nucleotide sequence of from about 20 to about 26 nucleotides which is selected from the nucleotide sequence of SEQ ID NO: 2.

43. The method according to claim 37, wherein the animal is administered an HDAC-1 antisense oligonucleotide that is SEQ ID NO: 2.

44. The method according to claim 18 or 30, further comprising administering to an animal a therapeutically effective amount of a small molecule inhibitor of HDAC-1.

45. The method according to claim 18 or 30, wherein an antisense oligonucleotide complementary to a portion of a nucleic acid encoding HDAC-7 and an antisense oligonucleotide complementary to a portion of a nucleic acid encoding HDAC-8 is administered.

46. The method according to claim 18 or 30, wherein a small molecule inhibitor of HDAC-7 and a small molecule inhibitor of HDAC-8 is administered.

47. The method according to claim 45, further comprising administering to the animal a therapeutically effective amount of an antisense oligonucleotide complementary to a region of RNA that encodes a portion of HDAC-1.

48. The method according to claim 45 further comprising administering to the animal a small molecule inhibitor of HDAC-1.

49. The method according to claim 46, further comprising administering to the animal a therapeutically effective amount of an antisense oligonucleotide complementary to a region of RNA that encodes a portion of HDAC-1.

50. The method according to claim 46 further comprising administering to the animal a small molecule inhibitor of HDAC-1.


SEQUENCE LISTING (41 sequences)

SEQ ID NO: 1 - DNA, 3131 bp, Homo sapiens
ataatacctaccttgcaggaccacgacaggattaagtgaggaaaaacccccatgagagtgttttgccattgtcaagtgagcctgagggaggctgaggggggatcaggctgtatcatgcccccgaggacaaactttccagtttaccctgctccctctctctgtccctaggctgccccaggccctgtgcagacacaccaggccctcagccgcagcccatggacctgcgggtgggccagcggcccccagtggagcccccaccagagcccacattgctggccctgcagcgtccccagcgcctgcaccaccacctcttcctagcaggcctgcagcagcagcgctcggtggagcccatgaggctctccatggacacgccgatgcccgagttgcaggtgggaccccaggaacaagagctgcggcagcttctccacaaggacaagagcaagcgaagtgctgtagccagcagcgtggtcaagcagaagctagcggaggtgattctgaaaaaacagcaggcggccctagaaagaacagtccatcccaacagccccggcattccctacagaaccctggagcccctggagacggaaggagccacccgctccatgctcagcagctttttgcctcctgttcccagcctgcccagtgaccccccagagcacttccctctgcgcaagacagtctctgagcccaacctgaagctgcgctataagcccaagaagtccctggagcggaggaagaatccactgctccgaaaggagagtgcgccccccagcctccggcggcggcccgcagagaccctcggagactcctccccaagtagtagcagcacgcccgcatcagggtgcagctcccccaatgacagcgagcacggccccaatcccatcctgggcgacagtgaccgcaggacccatccgactctgggccctcgggggccaatcctggggagcccccacactcccctcttcctgccccatggcttggagcccgaggctgggggcaccttgccctctcgcctgcagcccattctcctcctggacccctcaggctctcatgccccgctgctgactgtgcccgggcttgggcccttgcccttccactttgcccagtccttaatgaccaccgagcggctctctgggtcaggcctccactggccactgagccggactcgctcagagcccctgccccccagtgccaccgctcccccaccgccgggccccatgcagccccgcctggagcagctcaaaactcacgtccaggtgatcaagaggtcagccaagccgagtgagaagccccggctgcggcagataccctcggctgaagacctggagacagatggcgggggaccgggccaggtggtggacgatggcctggagcacagggagctgggccatgggcagcctgaggccagaggccccgctcctctccagcagcaccctcaggtgttgctctgggaacagcagcgactggctgggcggctcccccggggcagcaccggggacactgtgctgcttcctctggcccagggtgggcaccggcctctgtcccgggctcagtcttccccagccgcacctgcctcactgtcagccccagagcctgccagccaggcccgagtcctctccagctcagagacccctgccaggaccctgcccttcaccacagggctgatctatgactcggtcatgctgaagcaccagtgctcctgcggtgacaacagcaggcacccggagcacgccggccgcatccagagcatctggtcccggctgcaggagcgggggctccggagccagtgtgagtgtctccgaggccggaaggcctccctggaagagctgcagtcggtccactctgagcggcacgtgctcctctacggcaccaacccgctcagccgcctcaaactggacaacgggaagctggcagggctcctggcacagcggatgtttgagatgctgccctgtggtggggttggggtggacactgacaccatctggaatgagcttcattcctccaatgcagcccgctgggccgctggcagtgtcactgacctcgccttcaaagtggcttctcgtgagctaaagaatggtttcgctgtggtgcggcccccaggacaccatgcagatcattcaacagccatgggcttctgcttcttcaactcagtggccatcgcctgccggcagctgcaacagcagagcaaggccagcaaggccagcaagatcctcattgtagactgggacgtgcaccatggcaacggcacccagcaaaccttctaccaagaccccagtgtgctctacatctccctgcatcgccatgacgacggcaacttcttcccggggagtggggctgtggatgaggtaggggctggcagcggtgagggcttcaatgtcaatgtggcctgggctggaggtctggacccccccatgggggatcctgagtacctggctgctttcaggatagtcgtgatgcccatcgcccgagagttctctccagacctagtcctggtgtctgctggatttgatgctgctgagggtcacccggccccactgggtggctaccatgtttctgccaaatgttttggatacatgacgcagcaactgatgaacctggcaggaggcgcagtggtgctggccttggagggtggccatgacctcacagccatctgtgacgcctctgaggcctgtgtggctgctcttctgggtaacagggtggatcccctttcagaagaaggctggaaacagaaaccccaacctcaatgccatccgctctctggaggccgtgatccgggtgcacagtaaatactggggctgcatgcagcgcctggcctcctgtccagactcctgggtgcctagagtgccaggggctgacaaagaagaagtggaggcagtgaccgcactggcgtccctctctgtgggcatcctggctgaagataggccctcggagcagctggtggaggaggaagaacctatgaatctctaaggctctggaaccatctgcccgcccaccatgcccttgggacctggttctcttctaacccctggcaatagcccccattcctgggtctttagagatcctgtgggcaagtagttggaaccagagaacagcctgcctgctttgacagttatcccagggagcgtgagaaaatc

SEQ ID NO: 2 - DNA, 1654 bp, Homo sapiens
  modified_base (1590)..(1590): a, t, c or g
gaaattcggcacgagctcgtgccgaattcggcacgagaacggttttaagcggaagatggaggagccggaggaaccggcggacagtgggcagtcgctggtcccggtttatatctatagtcccgagtatgtcagtatgtgtgactccctggccaagatccccaaacgggccagtatggtgcattctttgattgaagcatatgcactgcataagcaaatgaggatagttaagcctaaagtggcctccatggaggagatggccaccttccacactgatgcttatctgcagcatctccagaaggtcagccaagagggcgatgatgatcatccggactccatagaatatgggctaggttatgactgcccagccactgaagggatatttgactatgcagcagctataggaggggctacgatcacagctgcccaatgcctgattgacggaatgtgcaaagtagcaattaactggtctggagggtggcatcatgcaaagaaagatgaagcatctggtttttgttatctcaatgatgctgtcctgggaatattacgattgcgacggaaatttgagcgtattctctacgtggatttggatctgcaccatggagatggtgtagaagacgcattcagtttcacctccaaagtcatgaccgtgtccctgcacaaattctccccaggatttttcccaggaacaggtgacgtgtctgatgttggcctagggaagggacggtactacagtgtaaatgtgcccattcaggatggcatacaagatgaaaaatattaccagatctgtgaaagtgtactaaaggaagtataccaagcctttaatcccaaagcagtggtcttacagctgggagctgacacaatagctggggatcccatgtgctcctttaacatgactccagtgggaattggcaagtgtcttaagtacatccttcaatggcagttggcaacactcattttgggaggaggaggctataaccttgccaacacggctcgatgctggacatacttgaccggggtcatcctagggaaaacactatcctctgagatcccagatcatgagtttttcacagcatatggtcctgattatgtgctggaaatcacgccaagctgccggccagaccgcaatgagccccaccgaatccaacaaatcctcaactacatcaaagggaatctgaagcatgtggtctagttgacagaaagagatcaggtttccagagctgaggagtggtgcctataatgaagacagcgtgtttatgcaagcagtttgtggaatttgtgactgcagggaaaatttgaaagaaattacttcctgaaaatttccaaggggcatcaagtggcagctggcttcctggggtgaagaggcaggcaccccagagtcctcaactggacctaggggaagaaggagatatcccacatttaaagttcttatttaaaaaaacacacacacacaaatgaaatttttaatctttgaaaattatttttaagcgaattggggaggggagtattttaatcatcttaaatgaaacagatcagaagctggatgagagcagtcaccagtttgtagggcaggaggcagctgacaggcagggntngggcctcnggaccanccangtggagccctgggagaganggtactgatcngcagactgggagg

SEQ ID NO: 3 - DNA, 20 bp, Artificial Sequence
  Description of Combined DNA/RNA Molecule Synthetic oligonucleotide
caggctcacttgacaauggc

SEQ ID NO: 4 - protein, 855 aa, Homo sapiens [sequence omitted]

SEQ ID NO: 5 - protein, 377 aa, Homo sapiens
MEEPEEPADSGQSLVPVYIYSPEYVSMCDSLAKIPKRASMVHSLIEAYALHKQMRIVKPKVASMEEMATFHTDAYLQHLQKVSQEGDDDHPDSIEYGLGYDCPATEGIFDYAAAIGGATITAAQCLIDGMCKVAINWSGGWHHAKKDEASGFCYLNDAVLGILRLRRKFERILYVDLDLHHGDGVEDAFSFTSKVMTVSLHKFSPGFFPGTGDVSDVGLGKGRYYSVNVPIQDGIQDEKYYQICESVLKEVYQAFNPKAVVLQLGADTIAGDPMCSFNMTPVGIGKCLKYILQWQLATLILGGGGYNLANTARCWTYLTGVILGKTLSSEIPDHEFFTAYGPDYVLEITPSCRPDRNEPHRIQQILNYIKGNLKHVV

SEQ ID NO: 6 - DNA, 1611 bp, Homo sapiens
atgtctggggtctctgcccgctggtgctgctgtctcccactcggtcatcctgagaacacagcctgagcgtctctgtcactcggggtagaccacgcggggaggcgagcaagatggcgcagacgcagggcacccggaggaaagtctgttactactacgacggggatgttggaaattactattatggacaaggccacccaatgaagcctcaccgaatccgcatgactcataatttgctgctcaactatggtctctaccgaaaaatggaaatctatcgccctcacaaagccaatgctgaggagatgaccaagtaccacagcgatgactacattaaattcttgcgctccatccgtccagataacatgtcggagtacagcaagcagatgcagagattcaacgttggtgaggactgtccagtattcgatggcctgtttgagttctgtcagttgtctactggtggttctgtggcaagtgctgtgaaacttaataagcagcagacggacatcgctgtgaattgggctgggggcctgcaccatgcaaagaagtccgaggcatctggcttctgttacgtcaatgatatcgtcttggccatcctggaactgctaaagtatcaccagagggtgctgtacattgacattgatattcaccatggtgacggcgtggaagaggccttctacaccacggaccgggtcatgactgtgtcctttcataagtatggagagtacttcccaggaactggggacctacgggatatcggggctggcaaaggcaagtattatgctgttaactacccgctccgagacgggattgatgacgagtcctatgaggccattttcaagccggtcatgtccaaagtaatggagatgttccagcctagtgcggtggtcttacagtgtggctcagactccctatctggggatcggttaggttgcttcaatctaactatcaaaggacacgccaagtgtgtggaatttgtcaagagctttaacctgcctatgctgatgctgggaggcggtggttacaccattcgtaacgttgcccggtgctggacatatgagacagctgtggccctggatacggagatccctaatgagcttccatacaatgactactttgaatactttggaccagatttcaagctccacatcagtccttccaatatgactaaccagaacacgaatgagtacctggagaagatcaaacagcgactgtttgagaaccttagaatgctgccgcacgcacctggggtccaaatgcaggcgattcctgaggacgccatccctgaggagagtggcgatgaggacgaagacgaccctgacaagcgcatctcgatctgctcctctgacaaacgaattgcctgtgaggaagagttctccgattctgaagaggagggagaggggggccgcaagaactcttccaacttcaaaaaagccaagagagtcaaaacagaggatgaaaaagagaaagacccagaggagaagaaagaagtcaccgaagaggagaaaaccaaggaggagaagccagaagccaaaggggtcaaggaggaggtcaagttggcctgaatggacctctccagctctggcttcctgctgagtccctcacgtttctttcccc

SEQ ID NO: 7 - protein, 482 aa, Homo sapiens
MAQTQGTRRKVCYYYDGDVGNYYYGQGHPMKPHRIRMTHNLLLNYGLYRKMEIYRPHKANAEEMTKYHSDDYIKFLRSIRPDNMSEYSKQMQRFNVGEDCPVFDGLFEFCQLSTGGSVASAVKLNKQQTDIAVNWAGGLHHAKKSEASGFCYVNDIVLAILELLKYHQRVLYIDIDIHHGDGVEEAFYTTDRVMTVSFHKYGEYFPGTGDLRDIGAGKGKYYAVNYPLRDGIDDESYEAIFKPVMSKVMEMFQPSAVVLQCGSDSLSGDRLGCFNLTIKGHAKCVEFVKSFNLPMLMLGGGGYTIRNVARCWTYETAVALDTEIPNELPYNDYFEYFGPDFKLHISPSNMTNQNTNEYLEKIKQRLFENLRMLPHAPGVQMQAIPEDAIPEESGDEDEDDPDKRISICSSDKRIACEEEFSDSEEEGEGGRKNSSNFKKAKRVKTEDEKEKDPEEKKEVTEEEKTKEEKPEAKGVKEEVKLA

SEQ ID NO: 8 - DNA, 20 bp, Artificial Sequence
  Description of Combined DNA/RNA Molecule Synthetic oligonucleotide
gaaacgtgagggactcagca

SEQ ID NO: 9 - DNA, 20 bp, Artificial Sequence
  Description of Combined DNA/RNA Molecule Synthetic oligonucleotide
guuaggtgaggcactgagga

SEQ ID NO: 10 - DNA, 20 bp, Artificial Sequence
  Description of Combined DNA/RNA Molecule Synthetic oligonucleotide
ggaagccagagctggagagg

SEQ ID NO: 11 - DNA, 20 bp, Artificial Sequence
  Description of Combined DNA/RNA Molecule Synthetic oligonucleotide
gcugagctgttctgatuugg

SEQ ID NO: 12 - DNA, 20 bp, Artificial Sequence
  Description of Combined DNA/RNA Molecule Synthetic oligonucleotide
cgugagcacttctcatuucc

SEQ ID NO: 13 - DNA, 20 bp, Artificial Sequence
  Description of Combined DNA/RNA Molecule Synthetic oligonucleotide
cgcuttccttgtcattgaca

SEQ ID NO: 14 - DNA, 20 bp, Artificial Sequence
  Description of Combined DNA/RNA Molecule Synthetic oligonucleotide
gccuttcctactcattgugu

SEQ ID NO: 15 - DNA, 20 bp, Artificial Sequence
  Description of Combined DNA/RNA Molecule Synthetic oligonucleotide
gguaccattgtcaggccuug

SEQ ID NO: 16 - DNA, 20 bp, Artificial Sequence
  Description of Combined DNA/RNA Molecule Synthetic oligonucleotide
ccuaccattcacaggccuac

SEQ ID NO: 17 - DNA, 20 bp, Artificial Sequence
  Description of Combined DNA/RNA Molecule Synthetic oligonucleotide
gcugcctgccgtgcccaccc

SEQ ID NO: 18 - DNA, 20 bp, Artificial Sequence
  Description of Combined DNA/RNA Molecule Synthetic oligonucleotide
cgugcctgcgctgcccacgg

SEQ ID NO: 19 - DNA, 20 bp, Artificial Sequence
  Description of Combined DNA/RNA Molecule Synthetic oligonucleotide
uacagtccatgcaaccucca

SEQ ID NO: 20 - DNA, 20 bp, Artificial Sequence
  Description of Combined DNA/RNA Molecule Synthetic oligonucleotide
aucagtccaaccaaccucgu

SEQ ID NO: 21 - DNA, 20 bp, Artificial Sequence
  Description of Combined DNA/RNA Molecule Synthetic oligonucleotide
gcagcggcggcagcaccucc

SEQ ID NO: 22 - DNA, 20 bp, Artificial Sequence
  Description of Combined DNA/RNA Molecule Synthetic oligonucleotide
cttcggtctcacctgcttgg

SEQ ID NO: 23 - DNA, 20 bp, Artificial Sequence
  Description of Combined DNA/RNA Molecule Synthetic oligonucleotide
cguugggagagttcatgccg

SEQ ID NO: 24 - DNA, 20 bp, Artificial Sequence
  Description of Combined DNA/RNA Molecule Synthetic oligonucleotide
caggctggaatgagctacag

SEQ ID NO: 25 - DNA, 20 bp, Artificial Sequence
  Description of Combined DNA/RNA Molecule Synthetic oligonucleotide
gacgctgcaatcaggtagac

SEQ ID NO: 26 - DNA, 20 bp, Artificial Sequence
  Description of Combined DNA/RNA Molecule Synthetic oligonucleotide
guggcacacaagacaauccc

SEQ ID NO: 27 - DNA, 20 bp, Artificial Sequence
  Description of Combined DNA/RNA Molecule Synthetic oligonucleotide
cuucagccaggatgcccaca

SEQ ID NO: 28 - DNA, 20 bp, Artificial Sequence
  Description of Combined DNA/RNA Molecule Synthetic oligonucleotide
cuccggctcctccatcuucc

SEQ ID NO: 29 - DNA, 20 bp, Artificial Sequence
  Description of Combined DNA/RNA Molecule Synthetic oligonucleotide
gaccggctgcaccatcttgg

SEQ ID NO: 30 - DNA, 20 bp, Artificial Sequence
  Description of Combined DNA/RNA Molecule Synthetic oligonucleotide
agccagctgccacttgaugc

SEQ ID NO: 31 - DNA, 20 bp, Artificial Sequence
  Description of Combined DNA/RNA Molecule Synthetic oligonucleotide
ucccagctggctcttgaagg

SEQ ID NO: 32 - DNA, 20 bp, Artificial Sequence
  Description of Combined DNA/RNA Molecule Synthetic oligonucleotide
nnnnnnnnnnnnnnnnnnnn

SEQ ID NO: 33 - DNA, 3036 bp, Homo sapiens
atgcacagtatgatcagctcagtggatgtgaagtcagaagttcctgtgggcctggagcccatctcacctttagacctaaggacagacctcaggatgatgatgcccgtggtggaccctgttgtccgtgagaagcaattgcagcaggaattacttcttatccagcagcagcaacaaatccagaagcagcttctgatagcagagtttcagaaacagcatgagaacttgacacggcagcaccaggctcagcttcaggagcatatcaaggaacttctagccataaaacagcaacaagaactcctagaaaaggagcagaaactggagcagcagaggcaagaacaggaagtagagaggcatcgcagagaacagcagcttcctcctctcagaggcaaagatagaggacgagaaagggcagtggcaagtacagaagtaaagcagaagcttcaagagttcctactgagtaaatcagcaacgaaagacactccaactaatggaaaaaatcattccgtgagccgccatcccaagctctggtacacggctgcccaccacacatcattggatcaaagctctccaccccttagtggaacatctccatcctacaagtacacattaccaggagcacaagatgcaaaggatgatttcccccttcgaaaaactgcctctgagcccaacttgaaggtgcggtccaggttaaaacagaaagtggcagagaggagaagcagccccttactcaggcggaaggatggaaatgttgtcacttcattcaagaagcgaatgtttgaggtgacagaatcctcagtcagtagcagttctccaggctctggtcccagttcaccaaacaatgggccaactggaagtgttactgaaaatgagacttcggttttgccccctacccctcatgccgagcaaatggtttcacagcaacgcattctaattcatgaagattccatgaacctgctaagtctttatacctctccttctttgcccaacattaccttggggcttcccgcagtgccatcccagctcaatgcttcgaattcactcaaagaaaagcagaagtgtgagacgcagacgcttaggcaaggtgttcctctgcctgggcagtatggaggcagcatcccggcatcttccagccaccctcatgttactttagagggaaagccacccaacagcagccaccaggctctcctgcagcatttattattgaaagaacaaatgcgacagcaaaagcttcttgtagctggtggagttcccttacatcctcagtctcccttggcaacaaaagagagaatttcacctggcattagaggtacccacaaattgccccgtcacagacccctgaaccgaacccagtctgcacctttgcctcagagcacgttggctcagctggtcattcaacagcaacaccagcaattcttggagaagcagaagcaataccagcagcagatccacatgaacaaactgctttcgaaatctattgaacaactgaagcaaccaggcagtcaccttgaggaagcagaggaagagcttcagggggaccaggcgatgcaggaagacagagcgccctctagtggcaacagcactaggagcgacagcagtgcttgtgtggatgacacactgggacaagttggggctgtgaaggtcaaggaggaaccagtggacagtgatgaagatgctcagatccaggaaatggaatctggggagcaggctgcttttatgcaacagcctttcctggaacccacgcacacacgtgcgctctctgtgcgccaagctccgctggctgcggttggcatggatggattagagaaacaccgtctcgtctccaggactcactcttcccctgctgcctctgttttacctcacccagcaatggaccgccccctccagcctggctctgcaactggaattgcctatgaccccttgatgctgaaacaccagtgcgtttgtggcaattccaccacccaccctgagcatgctggacgaatacagagtatctggtcacgactgcaagaaactgggctgctaaataaatgtgagcgaattcaaggtcgaaaagccagcctggaggaaatacagcttgttcattctgaacatcactcactgttgtatggcaccaaccccctggacggacagaagctggaccccaggatactcctaggtgatgactctcaaaagtttttttcctcattaccttgtggtggacttggggtggacagtgacaccatttggaatgagctacactcgtccggtgctgcacgcatggctgttggctgtgtcatcgagctggcttccaaagtggcctcaggagagctgaagaatgggtttgctgttgtgaggccccctggccatcacgctgaagaatccacagccatggggttctgcttttttaattcagttgcaattaccgccaaatacttgagagaccaactaaatataagcaagatattgattgtagatctggatgttcaccatggaaacggtacccagcaggccttttatgctgaccccagcatcctgtacatttcactccatcgctatgatgaagggaactttttccctggcagtggagccccaaatgaggttggaacaggccttggagaagggtacaatataaatattgcctggacaggtggccttgatcctcccatgggagatgttgagtaccttgaagcattcaggaccatcgtgaagcctgtggccaaagagtttgatccagacatggtcttagtatctgctggatttgatgcattggaaggccacacccctcctctaggagggtacaaagtgacggcaaaatgttttggtcatttgacgaagcaattgatgacattggctgatggacgtgtggtgttggctctagaaggaggacatgatctcacagccatctgtgatgcatcagaagcctgtgtaaatgcccttctaggaaatgagctggagccacttgcagaagatattctccaccaaagcccgaatatgaatgctgttatttctttacagaagatcattgaaattcaaagtatgtctttaaagttctcttaa

SEQ ID NO: 34 - protein, 1011 aa, Homo sapiens
MHSMISSVDVKSEVPVGLEPISPLDLRTDLRMMMPVVDPVVREKQLQQELLLIQQQQQIQKQLLIAEFQKQHENLTRQHQAQLQEHIKELLAIKQQQELLEKEQKLEQQRQEQEVERHRREQQLPPLRGKDRGRERAVASTEVKQKLQEFLLSKSATKDTPTNGKNHSVSRHPKLWYTAAHHTSLDQSSPPLSGTSPSYKYTLPGAQDAKDDFPLRKTASEPNLKVRSRLKQKVAERRSSPLLRRKDGNVVTSFKKRMFEVTESSVSSSSPGSGPSSPNNGPTGSVTENETSVLPPTPHAEQMVSQQRILIHEDSMNLLSLYTSPSLPNITLGLPAVPSQLNASNSLKEKQKCETQTLRQGVPLPGQYGGSIPASSSHPHVTLEGKPPNSSHQALLQHLLLKEQMRQQKLLVAGGVPLHPQSPLATKERISPGIRGTHKLPRHRPLNRTQSAPLPQSTLAQLVIQQQHQQFLEKQKQYQQQIHMNKLLSKSIEQLKQPGSHLEEAEEELQGDQAMQEDRAPSSGNSTRSDSSACVDDTLGQVGAVKVKEEPVDSDEDAQIQEMESGEQAAFMQQPFLEPTHTRALSVRQAPLAAVGMDGLEKHRLVSRTHSSPAASVLPHPAMDRPLQPGSATGIAYDPLMLKHQCVCGNSTTHPEHAGRIQSIWSRLQETGLLNKCERIQGRKASLEEIQLVHSEHHSLLYGTNPLDGQKLDPRILLGDDSQKFFSSLPCGGLGVDSDTIWNELHSSGAARMAVGCVIELASKVASGELKNGFAVVRPPGHHAEESTAMGFCFFNSVAITAKYLRDQLNISKILIVDLDVHHGNGTQQAFYADPSILYISLHRYDEGNFFPGSGAPNEVGTGLGEGYNINIAWTGGLDPPMGDVEYLEAFRTIVKPVAKEFDPDMVLVSAGFDALEGHTPPLGGYKVTAKCFGHLTKQLMTLADGRVVLALEGGHDLTAICDASEACVNALLGNELEPLAEDILHQSPNMNAVISLQKIIEIQSMSLKFS

SEQ ID NO: 35 - DNA, 2010 bp, Homo sapiens
atggggaccgcgcttgtgtaccatgaggacatgacggccacccggctgctctgggacgaccccgagtgcgagatcgagcgtcctgagcgcctgaccgcagccctggatcgcctgcggcagcgcggcctggaacagaggtgtctgcggttgtcagcccgcgaggcctcggaagaggagctgggcctggtgcacagcccagagtatgtatccctggtcagggagacccaggtcctaggcaaggaggagctgcaggcgctgtccggacagttcgacgccatctacttccacccgagtacctttcactgcgcgcggctggccgcaggggctggactgcagctggtggacgctgtgctcactggagctgtgcaaaatgggcttgccctggtgaggcctcccgggcaccatggccagagggcggctgccaacgggttctgcgtgttcaacaacgtggccatagcagctgcacatgccaagcagaaacacgggctacacaggatcctcgtcgtggactgggatgtgcaccatggccaggggatccagtatctctttgaggatgaccccagcgtcctttacttctcctggcaccgctatgagcatgggcgcttctggcctttcctgcgagagtcagatgcagacgcagtggggcggggacagggcctcggcttcactgtcaacctgccctggaaccaggttgggatgggaaacgctgactacgtggctgccttcctgcacctgctgctcccactggcctttgagtttgaccctgagctggtgctggtctcggcaggatttgactcagccatcggggaccctgaggggcaaatgcaggccacgccagagtgcttcgcccacctcacacagctgctgcaggtgctggccggcggccgggtctgtgccgtgctggagggcggctaccacctggagtcactggcggagtcagtgtgcatgacagtacagacgctgctgggtgacccggccccacccctgtcagggccaatggcgccatgtcagagtgccctagagtccatccagagtgcccgtgctgcccaggccccgcactggaagagcctccagcagcaagatgtgaccgctgtgccgatgagccccagcagccactccccagaggggaggcctccacctctgctgcctgggggtccagtgtgtaaggcagctgcatctgcaccgagctccctcctggaccagccgtgcctctgccccgcaccctctgtccgcaccgctgttgccctgacaacgccggatatcacattggttctgccccctgacgtcatccaacaggaagcgtcagccctgagggaggagacagaagcctgggccaggccacacgagtccctggcccgggaggaggccctcactgcacttgggaagctcctgtacctcttagatgggatgctggatgggcaggtgaacagtggtatagcagccactccagcctctgctgcagcagccaccctggatgtggctgttcggagaggcctgtcccacggagcccagaggctgctgtgcgtggccctgggacagctggaccggcctccagacctcgcccatgacgggaggagtctgtggctgaacatcaggggcaaggaggcggctgccctatccatgttccatgtctccacgccactgccagtgatgaccggtggtttcctgagctgcatcttgggcttggtgctgcccctggcctatggcttccagcctgacctggtgctggtggcgctggggcctggccatggcctgcagggcccccacgctgcactcctgactgcaatgcttcgggggctggcagggggccgagtcctggccctcctggaggagaactccacaccccagctagcagggatcctggcccgggtgctgaatggagaggcacctcctagcctaggcccttcctctgtggcctccccagaggacgtccaggccctgatgtacctgagagggcagctggagcctcagtggaagatgttgcagtgccatcctcacctggtggcttga

SEQ ID NO: 36 - protein, 669 aa, Homo sapiens
MGTALVYHEDMTATRLLWDDPECEIERPERLTAALDRLRQRGLEQRCLRLSAREASEEELGLVHSPEYVSLVRETQVLGKEELQALSGQFDAIYFHPSTFHCARLAAGAGLQLVDAVLTGAVQNGLALVRPPGHHGQRAAANGFCVFNNVAIAAAHAKQKHGLHRILVVDWDVHHGQGIQYLFEDDPSVLYFSWHRYEHGRFWPFLRESDADAVGRGQGLGFTVNLPWNQVGMGNADYVAAFLHLLLPLAFEFDPELVLVSAGFDSAIGDPEGQMQATPECFAHLTQLLQVLAGGRVCAVLEGGYHLESLAESVCMTVQTLLGDPAPPLSGPMAPCQSALESIQSARAAQAPHWKSLQQQDVTAVPMSPSSHSPEGRPPPLLPGGPVCKAAASAPSSLLDQPCLCPAPSVRTAVALTTPDITLVLPPDVIQQEASALREETEAWARPHESLAREEALTALGKLLYLLDGMLDGQVNSGIAATPASAAAATLDVAVRRGLSHGAQRLLCVALGQLDRPPDLAHDGRSLWLNIRGKEAAALSMFHVSTPLPVMTGGFLSCILGLVLPLAYGFQPDLVLVALGPGHGLQGPHAALLTAMLRGLAGGRVLALLEENSTPQLAGILARVLNGEAPPSLGPSSVASPEDVQALMYLRGQLEPQWKMLQCHPHLVA

SEQ ID NO: 37 - DNA, 1950 bp, Homo sapiens
atggggaccgcgcttgtgtaccatgaggacatgacggccacccggctgctctgggacgaccccgagtgcgagatcgagcgtcctgagcgcctgaccgcagccctggatcgcctgcggcagcgcggcctggaacagaggtgtctgcggttgtcagcccgcgaggcctcggaagaggagctgggcctggtgcacagcccagagtatgtatccctggtcagggagacccaggtcctaggcaaggaggagctgcaggcgctgtccggacagttcgacgccatctacttccacccgagtacctttcactgcgcgcggctggccgcaggggctggactgcagctggtggacgctgtgctcactggagctgtgcaaaatgggcttgccctggtgaggcctcccgggcaccatggccagagggcggctgccaacgggttctgcgtgttcaacaacgtggccatagcagctgcacatgccaagcagaaacacgggctacacaggatcctcgtcgtggactgggatgtgcaccatggccaggggatccagtatctctttgaggatgaccccagcgtcctttacttctcctggcaccgctatgagcatgggcgcttctggcctttcctgcgagagtcagatgcagacgcagtggggcggggacagggcctcggcttcactgtcaacctgccctggaaccaggttgggatgggaaacgctgactacgtggctgccttcctgcacctgctgctcccactggcctttgaggggcaaatgcaggccacgccagagtgcttcgcccacctcacacagctgctgcaggtgctggccggcggccgggtctgtgccgtgctggagggcggctaccacctggagtcactggcggagtcagtgtgcatgacagtacagacgctgctgggtgacccggccccacccctgtcagggccaatggcgccatgtcagagtgccctagagtccatccagagtgcccgtgctgcccaggccccgcactggaagagcctccagcagcaagatgtgaccgctgtgccgatgagccccagcagccactccccagaggggaggcctccacctctgctgcctgggggtccagtgtgtaaggcagctgcatctgcaccgagctccctcctggaccagccgtgcctctgccccgcaccctctgtccgcaccgctgttgccctgacaacgccggatatcacattggttctgccccctgacgtcatccaacaggaagcgtcagccctgagggaggagacagaagcctgggccaggccacacgagtccctggcccgggaggaggccctcactgcacttgggaagctcctgtacctcttagatgggatgctggatgggcaggtgaacagtggtatagcagccactccagcctctgctgcagcagccaccctggatgtggctgttcggagaggcctgtcccacggagcccagaggctgctgtgcgtggccctgggacagctggaccggcctccagacctcgcccatgacgggaggagtctgtggctgaacatcaggggcaaggaggcggctgccctatccatgttccatgtctccacgccactgccagtgatgaccggtggtttcctgagctgcatcttgggcttggtgctgcccctggcctatggcttccagcctgacctggtgctggtggcgctggggcctggccatggcctgcagggcccccacgctgcactcctgactgcaatgcttcgggggctggcagggggccgagtcctggccctcctggaggagaactccacaccccagctagcagggatcctggcccgggtgctgaatggagaggcacctcctagcctaggcccttcctctgtggcctccccagaggacgtccaggccctgatgtacctgagagggcagctggagcctcagtggaagatgttgcagtgccatcctcacctggtggcttga

SEQ ID NO: 38 - protein, 649 aa, Homo sapiens [sequence omitted]

SEQ ID NO: 39 - DNA, 30 bp, Artificial Sequence
  Description of Artificial Sequence Primer
cattcaggccaagtcgacctcctccttgac

SEQ ID NO: 40 - DNA, 41 bp, Artificial Sequence
  Description of Artificial Sequence Primer
atgaattcctgtgcacccggatcacggcctccagagagcgg

SEQ ID NO: 41 - DNA, 27 bp, Artificial Sequence
  Description of Artificial Sequence Primer
ccctcgaggaccacatgcttcagattc